SEQUENCE LISTING (37 sequences)

SEQ ID NO: 1 - DNA, 1380 bp, Artificial Sequence
  Synthetic Oligonucleotide
atggagtttgggctgagctgggttttcctcgttgctcttttaagaggtgtccagtgtcaggtgcagctggtggagtctgggggaggcgtggtccagcctgggaggtccctgagactctcctgtgtagcgtctggattcaccttcagtagccatggcatgcactgggtccgccaggctccaggcaaggggctggagtgggtggcagttatatggtatgatggaagaaataaatactatgcagactccgtgaagggccgattcaccatctccagagacaattccaagaacacgctgtttctgcaaatgaacagcctgagagccgaggacacggctgtgtattactgtgcgagaggaggtcacttcggtccttttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtct

SEQ ID NO: 2 - protein, 463 aa, Artificial Sequence
  Synthetic Peptide
MEFGLSWVFLVALLRGVQCQVQLVESGGGVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 167 aa, Artificial Sequence
  Synthetic Peptide
GVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSSASTKGPSVFPLAPCSRSTSESTAALGCLVKDYFPEPVTVSWNSGALTSGVHTFPAVLQ

SEQ ID NO: 4 - protein, 10 aa, Artificial Sequence
  Synthetic Peptide
GFTFSSHGMH

SEQ ID NO: 5 - protein, 15 aa, Artificial Sequence
  Synthetic Peptide
VIWYDGRNKYYADSV

SEQ ID NO: 6 - protein, 9 aa, Artificial Sequence
  Synthetic Peptide
GGHFGPFDY

SEQ ID NO: 7 - DNA, 708 bp, Artificial Sequence
  Synthetic Oligonucleotide
atggaaaccccagcgcagcttctcttcctcctgctactctggctcccagataccaccggagaaattgtgttgacgcagtctccaggcaccctgtctttgtctccaggggaaagagccaccctctcctgcagggccagtcagagtattagcagcagcttcttagcctggtaccagcagagacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtacctcaccctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 8 - protein, 235 aa, Artificial Sequence
  Synthetic Oligonucleotide
METPAQLLFLLLLWLPDTTGEIVLTQSPGTLSLSPGERATLSCRASQSISSSFLAWYQQRPGQAPRLLIYGASSRATGIPDRFSGSGSGTDFTLTISRLEPEDFAVYYCQQYGTSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 141 aa, Artificial Sequence
  Synthetic Oligonucleotide
QSPGTLSLSPGERATLSCRASQSISSSFLAWYQQRPGQAPRLLIYGASSRATGIPDRFSGSGSGTDFTLTISRLEPEDFAVYYCQQYGTSPWTFGQGTKVEIKRTVAAPSVFIFPPSDEQLKSGTASVVCLLNNFYPREAK

SEQ ID NO: 10 - protein, 12 aa, Artificial Sequence
  Synthetic Peptide
RASQSISSSFLA

SEQ ID NO: 11 - protein, 7 aa, Artificial Sequence
  Synthetic Peptide
GASSRAT

SEQ ID NO: 12 - protein, 10 aa, Artificial Sequence
  Synthetic Peptide
CQQYGTSPWT

SEQ ID NO: 13 - DNA, 1334 bp, Artificial Sequence
  Synthetic Oligonucleotide
caggtgcagctggtggagtctgggggaggcgtggtccagcctgggaggtccctgagactctcctgtgcagcgtctggattcaccttcagtagtcatggcatccactgggtccgccaggctccaggcaaggggctggagtgggtggcagttatatggtatgatggaagaaataaagactatgcagactccgtgaagggccgattcaccatctccagagacaattccaagaacacgctgtatttgcaaatgaacagcctgagagccgaggacacggctgtgtattactgtgcgagagtggccccactggggccacttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtgtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 444 aa, Artificial Sequence
  Synthetic Peptide
QVQLVESGGGVVQPGRSLRLSCAASGFTFSSHGIHWVRQAPGKGLEWVAVIWYDGRNKDYADSVKGRFTISRDNSKNTLYLQMNSLRAEDTAVYYCARVAPLGPLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 153 aa, Artificial Sequence
  Synthetic Peptide
PGRSLRLSCAASGFTFSSHGIHWVRQAPGKGLEWVAVIWYDGRNKDYADSVKGRFTISRDNSKNTLYLQMNSLRAEDTAVYYCARVAPLGPLDYWGQGTLVTVSSASTKGPSVFPLAPCSRSTSESTAALGCLVKDYFPEPVTVSWNSGALTS

SEQ ID NO: 16 - protein, 10 aa, Artificial Sequence
  Synthetic Peptide
GFTFSSHGIH

SEQ ID NO: 17 - protein, 15 aa, Artificial Sequence
  Synthetic Peptide
VIWYDGRNKDYADSV

SEQ ID NO: 18 - protein, 9 aa, Artificial Sequence
  Synthetic Peptide
VAPLGPLDY

SEQ ID NO: 19 - DNA, 645 bp, Artificial Sequence
  Synthetic Oligonucleotide
gaaattgtgttgacgcagtctccaggcaccctgtctttgtctccaggggaaagagccaccctctcctgcagggccagtcagagtgtcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaggattttgcagtgtattactgtcaacagtatggtaggtcaccattcactttcggccctgggaccaaagtagatatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 214 aa, Artificial Sequence
  Synthetic Peptide
EIVLTQSPGTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 146 aa, Artificial Sequence
  Synthetic Peptide
QSPGTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQWKVD

SEQ ID NO: 22 - protein, 11 aa, Artificial Sequence
  Synthetic Peptide
RASQSVSSYLA

SEQ ID NO: 23 - protein, 7 aa, Artificial Sequence
  Synthetic Peptide
GASSRAT

SEQ ID NO: 24 - protein, 10 aa, Artificial Sequence
  Synthetic Peptide
CQQYGRSPFT

SEQ ID NO: 25 - DNA, 1413 bp, Artificial Sequence
  Synthetic Oligonucleotide
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagatccgaggggagctaccctttactactactactacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - protein, 451 aa, Artificial Sequence
  Synthetic Peptide
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 167 aa, Artificial Sequence
  Synthetic Peptide
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 28 - protein, 10 aa, Artificial Sequence
  Synthetic Peptide
GFTFSSYGMH

SEQ ID NO: 29 - protein, 15 aa, Artificial Sequence
  Synthetic Peptide
VIWYDGSNKYYADSV

SEQ ID NO: 30 - protein, 16 aa, Artificial Sequence
  Synthetic Peptide
DPRGATLYYYYYGMDV

SEQ ID NO: 31 - DNA, 714 bp, Artificial Sequence
  Synthetic Oligonucleotide
atggacatgagggtccccgctcagctcctggggctcctgctactctggctccgaggtgccagatgtgacatccagatgacccagtctccatcctccctgtctgcatctgtaggagacagagtcaccatcacttgccgggcaagtcagagcattaacagctatttagattggtatcagcagaaaccagggaaagcccctaaactcctgatctatgctgcatccagtttgcaaagtggggtcccatcaaggttcagtggcagtggatctgggacagatttcactctcaccatcagcagtctgcaacctgaagattttgcaacttactactgtcaacagtattacagtactccattcactttcggccctgggaccaaagtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 32 - protein, 214 aa, Artificial Sequence
  Synthetic Peptide
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 139 aa, Artificial Sequence
  Synthetic Peptide
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 34 - protein, 11 aa, Artificial Sequence
  Synthetic Peptide
RASQSINSYLD

SEQ ID NO: 35 - protein, 7 aa, Artificial Sequence
  Synthetic Peptide
AASSLQS

SEQ ID NO: 36 - protein, 9 aa, Artificial Sequence
  Synthetic Peptide
QQYYSTPFT

SEQ ID NO: 37 - DNA, 24 bp, Artificial Sequence
  Synthetic Oligonucleotide
tcgtcgttttgtcgttttgtcgtt

(19) United States
(12) Patent Application Publication
(10) Pub. No.: US 2009/0117132 A1
(43) Pub. Date: May 7, 2009

(54) Anti-Ctla-4 Antibody and Cpg-Motif-Containing Synthetic Oligodeoxynucleotide Combination Therapy for Cancer Treatment

(75) Inventors: David Robert John Readett, Mystic, CT (US); Jarl Ulf Birger Jungnelius, Mystic, CT (US); Jesus Gomez-Navarro, Mystic, CT (US); Douglas Hanson, Niantic, CT (US); Arthur M. Krieg, Wellesley, MA (US)

Correspondence Address: PFIZER INC, 10555 SCIENCE CENTER DRIVE, SAN DIEGO, CA, 92121, US

(73) Assignees: Pfizer, Inc., New York, NY (US); Coley Pharmaceutical Group, Inc., Wellesley, MA (US)

(21) Appl. No.: 11/988,396

(22) PCT Filed: Jun. 30, 2006
(86) PCT No.: PCT/US2006/025711

§ 371 Date: Jan. 21, 2009

Related U.S. Application Data
(60) Provisional application No. 60/697,082, filed on Jul. 7, 2005.

(51) Int. Cl. A61K 39/395; A61P 35/00

(52) U.S. Cl. 424/172.1

(57) ABSTRACT

The invention relates to administration of an anti-CTLA-4 antibody, particularly human antibodies to human CTLA-4, such as those having amino acid sequences of antibodies 3.1.1, 4.1.1, 4.8.1, 4.10.2, 4.13.1, 4.14.3, 6.1.1, 11.2.1, 11.6.1, 11.7.1, 12.3.1.1, 12.9.1.1, and MDX-010, in combination with an immunostimulatory nucleotide, i.e, CpG ODN PF3512676, for treatment of cancer. The invention relates to administering a combination of an anti-CTLA-4 antibody and CpG ODN PF3512676 as neoadjuvant, adjuvant, first-line, second-line, and third-line therapy of cancer, whether localized or metastasized, and at any point(s) along the disease continuum (e.g, at any stage of the cancer).

### RELATED APPLICATIONS

This application claims priority to U.S. Provisional Application having Ser. No. 60/697,082, entitled “ANTI-CTLA-4 ANTIBODY AND CpG-MOTIF-CONTAINING SYNTHETIC OLIGODEOXYNUCLEOTIDE COMBINATION THERAPY FOR CANCER TREATMENT”, and filed on Jul. 7, 2005, the entire contents of which are incorporated by reference herein.

### FIELD OF THE INVENTION

The invention relates to the use of anti-CTLA-4 antibody in combination with CpG oligonucleotides for cancer treatment.

### BACKGROUND OF THE INVENTION

An alternative approach to cancer therapy is to target the immune system (“immunotherapy”) rather than and/or in addition to targeting the tumor itself. A potential benefit of immunotherapy is to provide improved efficacy by enhancing the patient's own immune response to tumors while minimizing deleterious effects to normal cells.

Cytotoxic T lymphocyte-associated antigen 4 (CTLA-4; CD152) is a cell surface receptor expressed on activated T cells. The natural ligands for CTLA-4 are B7.1 (CD80) and B7.2 (CD86), which are present on antigen-presenting cells (APCs, including dendritic cells, activated B-cells, and monocytes). CTLA-4 is a member of the immunoglobulin (Ig) superfamily of proteins that acts to down regulate T-cell activation and maintain immunologic homeostasis. In particular, it is believed that CD28 and CTLA-4 deliver opposing signals that are integrated by the T cell in determining the response to antigen. The outcome of T cell receptor stimulation by antigens is regulated by CD28 costimulatory signals, as well as inhibitory signals derived from CTLA-4. It is also determined by the interaction of CD28 or CTLA-4 on T cells with B7 molecules expressed on antigen presenting cells.

Experimental evidence indicates that binding of B7 to CTLA-4 delivers a negative regulatory signal to T cells, and that blocking this negative signal results in enhanced T cell immune function and antitumor activity in animal models (Thompson and Allison, 1997, Immunity 7:445-450; McCoy and LeGros, 1999, Immunol. & Cell Biol. 77:1-10). Several studies have demonstrated that treatment of mice with antimurine CTLA-4 blocking mAb markedly enhances T cell-mediated killing of various murine solid tumors, including established tumors, and can induce antitumor immunity (Leach et al., 1996, Science 271:1734-1736; Kwon et al., 1997, Proc. Natl. Acad. Sci. USA 94:8099-8103; Kwon et al., 1999, Proc. Natl. Acad. Sci. USA 96:15074-15079; Yang et al., 1997, Cancer Res. 57:4036-4041; U.S. Pat. No. 6,682,736, to Hanson et al.). Further, polymorphisms of CTLA-4 in humans have been associated with increased risk of autoimmune diseases such as rheumatoid arthritis and type I diabetes mellitus.

Additionally, U.S. Pat. No. 5,811,097 of Allison et al., refers to administration of CTLA-4 blocking agents to decrease tumor cell growth. International Publication No. WO 00/37504 (published Jun. 29, 2000) refers to human anti-CTLA-4 antibodies, and the use of those antibodies in treatment of cancer. WO 01/14424 (published Mar. 1, 2001) refers to additional human anti-CTLA-4 antibodies, and the use of such antibodies in treatment of cancer. WO 93/00431 (published Jan. 7, 1993) refers to regulation of cellular interactions with a monoclonal antibody reactive with a CTLA-4-Ig fusion protein. WO 00/32231 (published Jun. 8, 2000) refers to combination of a CTLA-4 blocking agent with a tumor vaccine to stimulate T-cells. WO 03/086459 refers to a method of promoting a memory response using CTLA-4 antibodies. Thus, the potential for development of therapeutics comprising inhibiting CTLA-4 binding to enhance and/or prolong an anti-tumor response has been demonstrated in the art.

Bacterial DNA has immune stimulatory effects to activate B cells and natural killer cells (Tokunaga, T., et al., 1988. Jpn. J. Cancer Res. 79:682-686; Tokunaga, T., et al., 1984, JNCI 72:955-962; Messina, J. P., et al., 1991, J. Immunol. 147:1759-1764; and reviewed in Krieg, 1998, In: Applied Oligonucleotide Technology, C. A. Stein and A. M. Krieg, (Eds.), John Wiley and Sons, Inc., New York, N.Y., pp. 431-448). The immune stimulatory effects of bacterial DNA are a result of the presence of unmethylated CpG dinucleotides in particular base contexts (CpG motifs), which are common in bacterial DNA, but methylated and underrepresented in vertebrate DNA (Krieg et al, 1995 Nature 374:546-549; Krieg, 1999 Biochim. Biophys. Acta 93321:1-10). The immune stimulatory effects of bacterial DNA can be mimicked with synthetic oligodeoxynucleotides (ODN) containing these CpG motifs. Such CpG ODN have highly stimulatory effects on human and murine leukocytes, inducing B cell proliferation, cytokine and immunoglobulin secretion, natural killer (NK) cell lytic activity, IFN-γ secretion, and activation of dendritic cells (DCs) and other antigen presenting cells to express costimulatory molecules and secrete cytokines, especially the Th1-like cytokines that are important in promoting the development of Th1-like T cell responses. The immune stimulatory effects of native phosphodiester backbone CpG ODN are highly CpG specific in that the effects are dramatically reduced if the CpG motif is methylated, changed to a GpC, or otherwise eliminated or altered (Krieg et al, 1995 Nature 374:546-549; Hartmann et al, 1999 Proc. Natl. Acad. Sci. USA 96:9305-10).

It was previously thought that the immune stimulatory effects required the CpG motif in the context of a purine-purine-CpG-pyrimidine-pyrimidine sequence (Krieg et al, 1995 Nature 374:546-549; Pisetsky, 1996 J. Immunol. 156:421-423; Hacker et al., 1998 EMBO J. 17:6230-6240; Lipford et al, 1998 Trends in Microbiol. 6:496-500). However, it is now clear that mouse lymphocytes respond quite well to phosphodiester CpG motifs not in this context (Yi et al., 1998 J. Immunol. 160:5898-5906) and the same is true of human B cells and dendritic cells (Hartmann et al, 1999 Proc. Natl. Acad. Sci. USA 96:9305-10; Liang, 1996 J. Clin. Invest. 98:1119-1129).

One class of CpG ODN is potent for activating B cells but is relatively weak in inducing IFN-α and NK cell activation; this class has been termed the B class. The B class CpG oligonucleotides typically are fully stabilized and include an unmethylated CpG dinucleotide within certain preferred base contexts. See, e.g., U.S. Pat. Nos. 6,194,388; 6,207,646; 6,214,806; 6,218,371; 6,239,116; and 6,339,068.

Although the individual use of anti-CTLA-4 antibodies or ODNs to induce an anti-tumor response hold great promise in the treatment of cancer, there remains a need to develop novel therapies to treat tumors, more particularly, solid tumors, with such immunotherapeutic approaches.

### SUMMARY OF THE INVENTION

Development of new therapeutic regimens, particularly those capable of augmenting or potentiating the anti-tumor activity of the immune system of the patient, while reducing the cytotoxic side effects of current chemotherapeutics, is necessary. The present invention provides such regimens.

Thus, in one embodiment, the invention provides a method for the treatment of cancer in a patient in need of such treatment, said method comprising administering to said patient a therapeutically effective amount of an anti-CTLA-4 antibody, or antigen-binding portion thereof, in combination with a therapeutically effective amount of CpG ODN PF3512676 (CpG 7909 (also known as ProMune); TCG TCG TTT TGT CGT TTT GTC GTT; SEQ ID NO:37). In one embodiment, the method is a non-vaccine method.

In one embodiment, said the CpG ODN is administered daily, every other day, twice a week, or weekly.

In one embodiment, said treatment is a therapy selected from the group consisting of neoadjuvant therapy, adjuvant therapy, first-line therapy, second-line therapy, and third-line therapy.

Depending on the embodiment, said cancer is selected from the group consisting of brain cancer, breast cancer, cervical cancer, colorectal carcinoma, cutaneous T-cell lymphoma, gastric cancer, head and neck cancer, liver cancer, lung cancer, melanoma, acute myeloid leukemia, Non-Hodgkin's lymphoma, ovarian cancer, pancreatic cancer, prostate cancer, renal cell carcinoma, and sarcoma.

In other embodiments, said therapeutically effective amount of said human anti-CTLA-4 antibody ranges from about 0.1 mg/kg to 50 mg/kg, or from about 0.3 mg/kg to 20 mg/kg, including but not limited to a therapeutically effective amount of said human anti-CTLA-4 antibody selected from the group consisting of at least 1 mg/kg, at least 3 mg/kg, at least 6 mg/kg, at least 10 mg/kg, and at least 15 mg/kg.

In one embodiment, said anti-CTLA-4 antibody, or antigen-binding portion thereof, is at least one antibody selected from the group consisting of (a) a human antibody having a binding affinity for CTLA-4 of about 10⁻⁸ or greater, and which inhibits binding between CTLA-4 and B7-1, and binding between CTLA-4 and B7-2; (b) a human antibody having an amino acid sequence comprising at least one human CDR sequence that corresponds to a CDR sequence from an antibody selected from the group consisting of 4.1.1, 4.8.1, 4.10.2, 4.13.1, 4.14.3, 6.1.1, 11.2.1, 11.6.1, 11.7.1., 12.3.1.1, 12.9.1.1, and 10D1; (c) a human antibody having the amino acid sequence of a heavy and/or light chain of an antibody selected from the group consisting of 4.1.1, 4.8.1, 4.10.2, 4.13.1, 4.14.3, 6.1.1, 11.2.1, 11.6.1, 11.7.1., 12.3.1.1, 12.9.1.1, and 10D1; (d) an antibody, or antigen-binding portion thereof, that competes for binding with CTLA-4 with at least one antibody having the amino acid sequence of an antibody selected from the group consisting of 4.1.1, 4.8.1, 4.10.2, 4.13.1, 4.14.3, 6.1.1, 11.2.1, 11.6.1, 11.7.1., 12.3.1.1, 12.9.1.1, and 10D1; and (e) an antibody, or antigen-binding portion thereof, that cross-competes for binding with CTLA-4 with at least one antibody having the amino acid of an antibody selected from the group consisting of 4.1.1, 4.8.1, 4.10.2, 4.13.1, 4.14.3, 6.1.1, 11.2.1, 11.6.1, 11.7.1., 12.3.1.1, 12.9.1.1, and 10D1.

In another embodiment, said antibody is a human antibody having the amino acid sequence of an antibody selected from the group consisting of 4.1.1, 4.13.1, 11.2.1, and 10D1. In related embodiments, said antibody, or antigen-binding portion thereof, comprises a heavy chain and a light chain wherein the amino acid sequences of the heavy chain variable domain of said heavy chain and the light chain variable domain of said light chain are selected from the group consisting of (a) the amino acid sequence of SEQ ID NO:3 and the amino acid sequence of SEQ ID NO:9; (b) the amino acid sequence of SEQ ID NO:15 and the amino acid sequence of SEQ ID NO:21; (c) the amino acid sequence of SEQ ID NO:27 and the amino acid sequence of SEQ ID NO:33; (d) the amino acid sequence encoded by the nucleic acid sequence of SEQ ID NO:1 and the amino acid sequence encoded by the nucleic acid sequence of SEQ ID NO:7; (e) the amino acid sequence encoded by the nucleic acid sequence of SEQ ID NO:13 and the amino acid sequence encoded by the nucleic acid sequence of SEQ ID NO:19; (f) the amino acid sequence encoded by the nucleic acid sequence of SEQ ID NO:25 and the amino acid sequence encoded by the nucleic acid sequence of SEQ ID NO:31; and (g) the amino acid sequence of a variable domain of antibody 10D1.

In another related embodiment, said antibody, or antigen-binding portion thereof, is an antibody selected from the group consisting of (a) an antibody comprising the amino acid sequences set forth in SEQ ID NO:4, SEQ ID NO:5, SEQ ID NO:6, SEQ ID NO:10, SEQ ID NO:11 and SEQ ID NO:12; (b) an antibody comprising the amino acid sequences set forth in SEQ ID NO:16, SEQ ID NO:17, SEQ ID NO:18, SEQ ID NO:22, SEQ ID NO:23 and SEQ ID NO:24; and (c) an antibody comprising the amino acid sequences set forth in SEQ ID NO:28, SEQ ID NO:29, SEQ ID NO:30, SEQ ID NO:34, SEQ ID NO:35 and SEQ ID NO:36.

In yet another related embodiment, said antibody, or antigen-binding portion thereof, comprises a heavy chain variable region having the amino acid sequence set forth in SEQ ID NO:27 and a light chain variable region having the amino acid sequence set forth in SEQ ID NO:33.

In still another related embodiment, said antibody is selected from the group consisting of (a) an antibody comprising the amino acid sequences set forth in SEQ ID NO:2 and SEQ ID NO:8; (b) an antibody comprising the amino acid sequences set forth in SEQ ID NO:14 and SEQ ID NO:20; and (c) an antibody comprising the amino acid sequences set forth in SEQ ID NO:26 and SEQ ID NO:32.

In one embodiment, said antibody is administered 1-7 days prior to administration of said CpG ODN. In this and other embodiments, said CpG ODN is administered from about one to one-hundred days after said antibody.

In one embodiment, said CpG ODN is administered subcutaneously.

In another embodiment, said CpG ODN is administered in an amount of 1 mg-50 mg per day.

In another aspect, the invention provides a pharmaceutical composition for treatment of cancer, said composition comprising a therapeutically effective amount of an anti-CTLA-4 antibody, or antigen-binding portion thereof, and a therapeutically effective amount of CpG ODN PF3512676, and a pharmaceutically acceptable carrier.

These and other embodiments of the invention will be described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE DRAWINGS

The foregoing summary, as well as the following detailed description of the invention, will be better understood when read in conjunction with the appended drawings. For the purpose of illustrating the invention the drawings show embodiment(s) which are presently preferred. It should be understood, however, that the invention is not limited to the precise arrangements and instrumentalities shown.

In the drawings:

FIG. 1, comprising FIGS. 1A-1D, shows the nucleotide and amino acid sequences of anti-CTLA-4 antibody 4.1.1. FIG. 1A shows the full length nucleotide sequence for the 4.1.1 heavy chain (SEQ ID NO:1). FIG. 1B shows the full length amino acid sequence for the 4.1.1 heavy chain (SEQ ID NO:2), and the amino acid sequence for the 4.1.1 heavy chain variable region (SEQ ID NO:3) designated between brackets “[ ]”. The amino acid sequence of each 4.1.1 heavy chain CDR is underlined. The CDR sequences are as follows: CDR1: GFTFSSHGMH (SEQ ID NO:4); CDR2: VIWYDGRNKYYADSV (SEQ ID NO:5); and CDR3: GGHFGPFDY (SEQ ID NO:6). FIG. 1C shows the nucleotide sequence for the 4.1.1 light chain (SEQ ID NO:7). FIG. 1D shows the amino acid sequence of the full length 4.1.1 light chain (SEQ ID NO:8), and the variable region as indicated between brackets “[ ]” (SEQ ID NO:9). The amino acid sequence of each CDR is indicated as follows: CDR1: RASQSISSSFLA (SEQ ID NO:10); CDR2: GASSRAT (SEQ ID NO:11); and CDR3: CQQYGTSPWT (SEQ ID NO:12).

FIG. 2, comprising FIGS. 2A-2D, shows the nucleotide and amino acid sequences of anti-CTLA-4 antibody 4.13.1. FIG. 2A shows the full length nucleotide sequence for the 4.13.1 heavy chain (SEQ ID NO:13). FIG. 2B shows the full length amino acid sequence for the 4.13.1 heavy chain (SEQ ID NO:14), and the amino acid sequence for the 4.13.1 heavy chain variable region (SEQ ID NO:15) designated between brackets “[ ]”. The amino acid sequence of each 4.13.1 heavy chain CDR is underlined. The CDR sequences are as follows: CDR1: GFTFSSHGIH (SEQ ID NO:16); CDR2: VIWYDGRNKDYADSV (SEQ ID NO:12); and CDR3: VAPLGPLDY (SEQ ID NO:18). FIG. 2C shows the nucleotide sequence for the 4.13.1 light chain (SEQ ID NO:19). FIG. 2D shows the amino acid sequence of the full length 4.13.1 light chain (SEQ ID NO:20), and the variable region as indicated between brackets “[ ]” (SEQ ID NO:21). The amino acid sequence of each CDR is indicated as follows: CDR1: RASQSVSSYLA (SEQ ID NO:22); CDR2: GASSRAT (SEQ ID NO:23); and CDR3: CQQYGRSPFT (SEQ ID NO:24).

FIG. 3, comprising FIGS. 3A-3D, shows the nucleotide and amino acid sequences of anti-CTLA-4 antibody 11.2.1. FIG. 3A shows the full length nucleotide sequence for the 11.2.1 heavy chain (SEQ ID NO:25). FIG. 3B shows the full length amino acid sequence for the 11.2.1 heavy chain (SEQ ID NO:26), and the amino acid sequence for the 11.2.1 heavy chain variable region (SEQ ID NO:27) designated between brackets “[ ]”. The amino acid sequence of each 11.2.1 heavy chain CDR is underlined. The CDR sequences are as follows: CDR1: GFTFSSYGMH (SEQ ID NO:28); CDR2: VIWYDGSNKYYADSV (SEQ ID NO:29); and CDR3: DPRGATLYYYYYGMDV (SEQ ID NO:30). FIG. 3C shows the nucleotide sequence for the 11.2.1 light chain (SEQ ID NO:31). FIG. 3D shows the amino acid sequence of the full length 11.2.1 light chain (SEQ ID NO:32), and the variable region as indicated between brackets “[ ]” (SEQ ID NO:33). The amino acid sequence of each CDR is indicated as follows: CDR1: RASQSINSYLD (SEQ ID NO:34); CDR2: AASSLQS (SEQ ID NO:35); and CDR3: QQYYSTPFT (SEQ ID NO:36).

### DETAILED DESCRIPTION OF THE INVENTION

The invention relates to novel therapeutic methods comprising co-administering a combination of an anti-CTLA-4 antibody and a CpG ODN (i.e., CpG ODN PF3512676), for treatment of cancer. Cancers to be treated according to the invention include but are not limited to bladder cancer, brain tumors, breast cancer, cervical cancer, colorectal cancer, gastrointestinal cancer, head and neck cancer, hepatocellular carcinoma, Hodgkin's disease, Kaposi's sarcoma, acute and chronic leukemias, cutaneous T-cell leukemia, myeloid and lymphoid leukemias, lung cancer (including non-small cell lung carcinoma), melanoma, Non-Hodgkin's Lymphoma, ovarian cancer, pancreatic cancer, prostate cancer, renal cell carcinoma, squamous cell carcinoma of the skin, thyroid cancer, and carcinomas and sarcomas of other types (e.g., liposarcoma, osteosarcoma) among many others. In various embodiments, the method comprises administering CpG ODN PF3512676 in combination with the antibody for neoadjuvant, adjuvant, first-line, second-line, or third-line therapy for cancer.

Antibodies employable in the present invention, and methods of producing them, are described in the International Application No. PCT/US99/30895, published on Jun. 29, 2000 as WO 00/37504, European Patent Appi. No. EP 1262193 A1, published Apr. 12, 2002, U.S. patent application Ser. No. 09/472,087, now issued as U.S. Pat. No. 6,682,736, U.S. patent application Ser. No. 09/948,939, now published as U.S. Pat. App. Pub. No. 2002/0086014 (e.g., MDX-010, Medarex, Princeton, N.J.), each of which is incorporated by reference herein in its entirety. While information on the amino and nucleic acid sequences relating to these antibodies is provided herein, further information can be found in U.S. Pat. No. 6,682,736, as well as published applications WO 00/37504, EP 1262193, and US2002/0086014; the sequences set forth in those applications are hereby incorporated herein by reference.

Certain uses for these antibodies to treat various cancers were discussed in U.S. patent application Ser. No. 10/153,382, now published as U.S. Patent Application Publication No. 2003/0086930, which is incorporated by reference as if set forth in its entirety herein.

The CpG immunostimulatory oligonucleotide used in the present invention is a B class CpG immunostimulatory oligonucleotide. B class CpG immunostimulatory oligonucleotides have been described in U.S. Pat. Nos. 6,194,388 B1 and 6,239,116 B1, issued on Feb. 27, 2001 and May 29, 2001 respectively. The CpG immunostimulatory oligonucleotide of the invention is termed CpG ODN PF3512676 and it is defined by the following nucleotide sequence

(SEQ ID NO:37)

5′ TCG TCG TTT TGT CGT TTT GTC GTT 3′.

CpG ODN PF3512676 strongly activates human B cells and has minimal effects on interferon-α induction. As described in greater detail herein, CpG ODN PF3512676 may have a homogenous or a chimeric backbone, including but not limited to phosphodiester and phosphorothioate backbone linkages.

In another embodiment, the antibody-CpG ODN PF3512676 combination is administered with at least one additional therapeutic agent, such as, but not limited to other monoclonal antibodies not directed to CTLA-4 (e.g., AVASTIN (bevacizumab), MYELOTARG (gemtuzumab), BEXXAR (tositumomab), RITUXAN (rituximab), HERCEPTIN (trastuzumab)), or protein ligands having similar effects; agents that activate antigen presenting cells (dendritic cells, macrophages, B cells, monocytes), including type 1 interferons (e.g., interferon alpha and beta); interferon gamma; BCG; agents that provide tumor antigens in any and all forms, including protein antigens, peptide antigens, whole cell lysates and derivatives thereof; genetically encoded antigens (e.g., adenovirus encoded antigens); cellular components of the immune system that have been altered either in vivo or ex vivo to enhance their immune properties (e.g., autologous dendritic cells, lymphocytes, heat shock proteins, etc.); chemotherapeutic agents such as, but not limited to, cyclophosphamide, methotrexate, etoposide, adriamycin, taxanes, fluorouracil, cytosine arabinoside (AraC), and platinum-containing agents, among numerous others. Examples of antigens include PSA antigens (e.g., PROSTVAC/TRICOM) and melanoma-derived gp100 antigens. The combination may also be administered in combination with a cytokine or growth factor such as but not limited to GM-CSF.

In one embodiment, the method of treatment is a non-vaccine method. As used herein, a non-vaccine method means that the combination of CpG ODN PF3512676 and anti-CTLA-4 antibody is not used together with an exogenous antigen in order to stimulate an immune response to the antigen. A non-vaccine method however may encompass stimulating immune responses to endogenous antigens. Endogenous antigens include those expressed, released or shed by a cancer cell or mass in vivo.

### I. DEFINITIONS

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art.

The methods and techniques of the present invention are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference.

Conventional notation is used herein to portray polypeptide sequences: the left-hand end of a polypeptide sequence is the amino-terminus; the right-hand end of a polypeptide sequence is the carboxyl-terminus.

A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, Methods Mol. Biol. 243:307-31 (1994).

Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science 256:1443-45 (1992), herein incorporated by reference. A “moderately conservative” replacement is any change having a nonnegative value in the PAM250 log-likelihood matrix.

Preferred amino acid substitutions are those which: (1) reduce susceptibility to proteolysis, (2) reduce susceptibility to oxidation, (3) alter binding affinity for forming protein complexes, and (4) confer or modify other physicochemical or functional properties of such analogs. Analogs comprising substitutions, deletions, and/or insertions can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al., Nature 354:105 (1991), which are each incorporated herein by reference.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters. See, e.g., Altschul et al., J. Mol. Biol. 215:403-410 (1990); Altschul et al., Nucleic Acids Res. 25:3389-402 (1997); herein incorporated by reference.

An intact “antibody” comprises at least two heavy (H) chains and two light (L) chains inter-connected by disulfide bonds. See generally, Fundamental Immunology, Ch. 7 (Paul, W., ed., 2nd ed. Raven Press, N.Y. (1989)) (incorporated by reference in its entirety for all purposes). Each heavy chain is comprised of a heavy chain variable region (HCVR or V_{H}) and a heavy chain constant region (C_{H}). The heavy chain constant region is comprised of three domains, CH1, CH2 and CH3. Each light chain is comprised of a light chain variable region (LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The assignment of amino acids to each domain is in accordance with the definitions of Kabat, Sequences of Proteins of Immunological Interest (National Institutes of Health, Bethesda, Md. (1987 and 1991)), or Chothia & Lesk, J. Mol. Biol. 196:901-917 (1987); Chothia et al., Nature 342:878-883 (1989).

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antibody” can include antigen-binding portions of an intact antibody that retain capacity to specifically bind the antigen of the intact antibody, e.g., CTLA-4. Antigen-binding portions may be produced by recombinant DNA techniques or by enzymatic or chemical cleavage of intact antibodies.

Examples of antigen-binding portions include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a single domain antibody (“dAb”), which consists of a VH domain as described in Ward et al., Nature 341:544-546 (1989); and (vi) an isolated complementarity determining region (CDR). Furthermore, although the two domains of the Fv fragment, V_{H} and V_{L}, are coded for by separate genes, they can be joined, using recombinant methods, by a synthetic linker that enables them to be made as a single protein chain in which the V_{H} and V_{L} regions pair to form monovalent molecules (known as single chain Fv (scFv); See, e.g., Bird et al. Science 242:423-426 (1988); and Huston et al. Proc. Natl. Acad. Sci. USA 85:5879-5883 (1988)). Such single chain antibodies are included by reference to the term “antibody”.

A “bispecific antibody” has two different binding specificities, see, e.g., U.S. Pat. No. 5,922,845 and U.S. Pat. No. 5,837,243; Zeilder J. Immunol. 163:1246-1252 (1999); Somasundaram Hum. Antibodies 9:47-54 (1999); Keler Cancer Res. 57:4008-4014 (1997). For example, the invention provides bispecific antibodies having one binding site for a cell surface antigen, such as human CTLA-4, and a second binding site for an Fc receptor on the surface of an effector cell. The invention also provides multispecific antibodies, which have at least three binding sites.

The term “bispecific antibodies” further includes “diabodies.” Diabodies are bivalent, bispecific antibodies in which the V_{H} and V_{L} domains are expressed on a single polypeptide chain, but using a linker that is too short to allow for pairing between the two domains on the same chain, thereby forcing the domains to pair with complementary domains of another chain and creating two antigen binding sites (See, e.g., Holliger et al., Proc. Natl. Acad. Sci. USA 90:6444-6448 (1993); PoIjak et al., Structure 2:1121-1123 (1994)).

The terms “human antibody” or “human sequence antibody”, as used interchangeably herein, include antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include “chimeric” antibodies in which CDR sequences derived from the germline of another mammalian species, such as a mouse, have been grafted onto human framework sequences (i.e., “humanized” or PRIMATIZED™ antibodies).

The term “chimeric antibody” as used herein means an antibody that comprises regions from two or more different antibodies. In one embodiment, one or more of the CDRs are derived from a human anti-CTLA-4 antibody. In another embodiment, all of the CDRs are derived from a human anti-CTLA-4 antibody. In another embodiment, the CDRs from more than one human anti-CTLA-4 antibodies are combined in a chimeric human antibody. For instance, a chimeric antibody may comprise a CDR1 from the light chain of a first human anti-CD40 antibody, a CDR2 from the light chain of a second human anti-CTLA-4 antibody and a CDR3 and CDR3 from the light chain of a third human anti-CTLA-4 antibody, and the CDRs from the heavy chain may be derived from one or more other anti-CD40 antibodies. Further, the framework regions may be derived from one of the same anti-CTLA-4 antibodies or from one or more different human(s).

Moreover, as discussed previously herein, chimeric antibody includes an antibody comprising a portion derived from the germline sequences of more than one species.

By the term “compete”, as used herein with regard to an antibody, is meant that a first antibody, or an antigen-binding portion thereof, competes for binding with a second antibody, or an antigen-binding portion thereof, where binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). For instance, cross-competing antibodies can bind to the epitope, or potion of the epitope, to which the antibodies of the invention (e.g., 3.1.1, 4.1.1, 4.8.1, 4.10.2, 4.13.1, 4.14.3, 6.1.1, 11.2.1, 11.6.1, 11.7.1, 12.3.1.1, and 12.9.1.1) bind. Both competing and cross-competing antibodies are encompassed by the present invention. Regardless of the mechanism by which such competition or cross-competition occurs (e.g., steric hindrance, conformational change, or binding to a common epitope, or portion thereof, and the like), the skilled artisan would appreciate, based upon the teachings provided herein, that such competing and/or cross-competing antibodies are encompassed and can be useful for the methods disclosed herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

By the phrase “specifically binds,” as used herein, is meant a compound, e.g., a protein, a nucleic acid, an antibody, and the like, which recognizes and binds a specific molecule, but does not substantially recognize or bind other molecules in a sample. For instance, an antibody or a peptide inhibitor which recognizes and binds a cognate ligand (e.g., an anti-CTLA-4 antibody that binds with its cognate antigen, CTLA-4) in a sample, but does not substantially recognize or bind other molecules in the sample. Thus, under designated assay conditions, the specified binding moiety (e.g., an antibody or an antigen-binding portion thereof) binds preferentially to a particular target molecule and does not bind in a significant amount to other components present in a test sample. A variety of assay formats may be used to select an antibody that specifically binds a molecule of interest. For example, solid-phase ELISA immunoassay, immunoprecipitation, BIAcore and Western blot analysis are used to identify an antibody that specifically reacts with CTLA-4. Typically a specific or selective reaction will be at least twice background signal or noise and more typically more than 10 times background, even more specifically, an antibody is said to “specifically bind” an antigen when the equilibrium dissociation constant (K_{D}) is ≦1 μM, preferably ≦100 nM and most preferably ≦10 nM.

The term “K_{D}” refers to the equilibrium dissociation constant of a particular antibody-antigen interaction.

As used herein, “substantially pure” means an object species is the predominant species present (i.e., on a molar basis it is more abundant than any other individual species in the composition), and preferably a substantially purified fraction is a composition wherein the object species (e.g., an anti-CTLA-4 antibody) comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

By the term “effective amount”, or “therapeutically effective amount,” as used herein, is meant an amount that when administered to a mammal, preferably a human, mediates a detectable therapeutic response compared to the response detected in the absence of the compound. A therapeutic response, such as, but not limited to, inhibition of and/or decreased tumor growth (including tumor size stasis), tumor size, metastasis, and the like, can be readily assessed by a plethora of art-recognized methods, including, e.g., such methods as disclosed herein.

The skilled artisan would understand that the effective amount of the compound or composition administered herein varies and can be readily determined based on a number of factors such as the disease or condition being treated, the stage of the disease, the age and health and physical condition of the mammal being treated, the severity of the disease, the particular compound being administered, and the like.

A “therapeutic effective amount”, or “effective amount,” is intended to qualify the amount of an agent required to detectably reduce to some extent one or more of the symptoms of a neoplasia disorder, including, but is not limited to: 1) reduction in the number of cancer cells; 2) reduction in tumor size; 3) inhibition (i.e., slowing to some extent, preferably stopping) of cancer cell infiltration into peripheral organs; 3) inhibition (i.e., slowing to some extent, preferably stopping) of tumor metastasis; 4) inhibition, to some extent, of tumor growth; 5) relieving or reducing to some extent one or more of the symptoms associated with the disorder; and/or 6) relieving or reducing the side effects associated with the administration of anticancer agents.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the severity of the disease or condition, and the health and size of the subject. One of ordinary skill in the art can empirically determine the effective amount of CpG ODN PF3512676, anti-CTLA-4 antibodies, and/or other therapeutic agent without necessitating undue experimentation.

The therapeutically effective amount of ODN and/or antibodies alone or together can be initially determined from animal models. A therapeutically effective dose can also be determined from human data for the specific ODN and/or specific antibodies or for other compounds which are known to exhibit similar pharmacological activities. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

“Instructional material,” as that term is used herein, includes a publication, a recording, a diagram, or any other medium of expression which can be used to communicate the usefulness of the compound, combination, and/or composition of the invention in the kit for affecting, alleviating or treating the various diseases or disorders recited herein. Optionally, or alternately, the instructional material can describe one or more methods of alleviating the diseases or disorders in a cell, a tissue, or a mammal, including as disclosed elsewhere herein.

The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively.

The ODN and/or antibody of the invention may be provided in a medicinal dispenser. A medical dispenser is a package defining a plurality of medicinal storage compartments, each compartment for housing an individual unit of medicament. An entire medicinal course of treatment is housed in a plurality of medicinal storage compartments.

A package defining a plurality of medicinal storage compartments may be any type of disposable pharmaceutical package or card which holds medicaments in individual compartments. For example, the package is a blister package constructed from a card, which may be made from stiff paper material, a blister sheet and backing sheet. Such cards are well known to those of ordinary skill in the art.

As an example, a medicinal dispenser may house an entire medicinal course of treatment. The dispenser may include the day indicia to indicate which day the individual units of medicament are to be taken. These may be marked along a first side of the medicinal package. The dose indicia may also be marked, for example along a second side of the medicinal package perpendicular to the first side of the medicinal package, thereby indicating the time which the individual unit of medicament should be taken. The unit doses may be contained in the dispenser which is a blister pack.

Except when noted, the terms “patient” or “subject” are used interchangeably and refer to mammals such as human patients and non-human primates, as well as veterinary subjects such as rabbits, rats, and mice, and other animals. Preferably, patient refers to a human.

As used herein, to “treat” means reducing the frequency with which symptoms of a disease (i.e., tumor growth and/or metastasis, or other effect mediated by the numbers and/or activity of immune cells, and the like) are experienced by a patient. The term includes the administration of the compounds or agents of the present invention to prevent or delay the onset of the symptoms, complications, or biochemical indicia of a disease (e.g., elevation of PSA level in prostate cancer), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

“Combination therapy” embraces the administration of a CpG ODN PF3512676 and a CTLA-4 antibody as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention.

“Combination therapy” embraces administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular, subcutaneous routes, and direct absorption through mucous membrane tissues. The therapeutic agents can be administered by the same route or by different routes. For example, a first therapeutic agent (e.g., CpG ODN PF3512676) can be administered by subcutaneous injection, and a second agent (e.g., anti-CTLA-4 antibody) can be administered intravenously. Further, a first therapeutic agent of the combination selected may be administered by intravenous injection while the other therapeutic agents of the combination may be administered orally. Alternatively, for example, both the therapeutic agents may be administered orally or both therapeutic agents may be administered by intravenous injection.

“Combination therapy” also can embrace the administration of the therapeutic agents as described above in further combination with other biologically active ingredients (such as, but not limited to, a second and different antineoplastic agent, a dendritic vaccine or other tumor vaccine) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

### II. Anti-CTLA-4 Antibodies

As stated previously elsewhere herein, the preferred anti-CTLA-4 antibody is a human antibody that specifically binds to human CTLA-4. Exemplary human anti-CTLA-4 antibodies are described in detail in International Application No. PCT/US99/30895, published on Jun. 29, 2000 as WO 00/37504, European Patent Appl. No. EP 1262193 A1, published Apr. 12, 2002, and U.S. patent application Ser. No. 09/472,087, now issued as U.S. Pat. No. 6,682,736, to Hanson et al., as well as U.S. patent application Ser. No. 09/948,939, published as US2002/0086014, the entire disclosure of which is hereby incorporated by reference. Such antibodies include, but are not limited to, 3.1.1, 4.1.1, 4.8.1, 4.10.2, 4.13.1, 4.14.3, 6.1.1, 11.2.1, 11.6.1, 11.7.1, 12.3.1.1, and 12.9.1.1, as well as MDX-010. Human antibodies provide a substantial advantage in the treatment methods of the present invention, as they are expected to minimize the immunogenic and allergic responses that are associated with use of non-human antibodies in human patients.

Characteristics of useful human anti-CTLA-4 antibodies of the invention are extensively discussed in WO 00/37504, EP 1262193, and U.S. Pat. No. 6,682,736 as well as U.S. Patent Application Publication Nos. US2002/0086014 and US2003/0086930, and the amino and nucleic acid sequences set forth therein are incorporated by reference herein in their entirety. Briefly, the antibodies of the invention include antibodies having amino acid sequences of an antibody such as, but not limited to, antibody 3.1.1, 4.1.1, 4.8.1, 4.10.2, 4.13.1, 4.14.3, 6.1.1, 11.2.1, 11.6.1, 11.7.1, 12.3.1.1, 12.9.1.1, and MDX-010. The invention also relates to antibodies having the amino acid sequences of the CDRs of the heavy and light chains of these antibodies, as well as those having changes in the CDR regions, as described in the above-cited applications and patent. The invention also concerns antibodies having the variable regions of the heavy and light chains of those antibodies. In another embodiment, the antibody is selected from an antibody having the full length, variable region, or CDR, amino acid sequences of the heavy and light chains of antibodies 3.1.1, 4.1.1, 4.8.1, 4.10.2, 4.13.1, 4.14.3, 6.1.1, 11.2.1, 11.6.1, 11.7.1, 12.3.1.1, and 12.9.1.1, and MDX-010.

In one embodiment, the invention comprises an antibody-therapeutic agent combination comprising a human anti-CTLA-4 antibody disclosed in U.S. patent application Ser. No. 09/948,939, published as U.S. Patent Application Publication No. 2002/0086014 and No. 2003/0086930, and references cited therein, including, but not limited to, MAb 10D1 (MDX-010, Medarex, Princeton, N.J.). Even more preferably, the anti-CTLA-4 antibody is MDX-010. Alternatively, the anti-CTLA-4 antibody is 11.2.1 (Ticilimumab; CP-675,206).

In another embodiment, the amino acid sequence of the V_{H} comprises the amino acid sequences set forth in SEQ ID NOs:3, 15 and 27. In yet another embodiment, the V_{L} comprises the amino acid sequences set forth in SEQ ID NOs:9, 21 and 33. More preferably, the V_{H} and V_{L} comprise the amino acid sequences set forth in SEQ ID NO:3 (V_{H} 4.1.1) and SEQ ID NO:9 (V_{L} 4.1.1), respectively; the amino acid sequences set forth in SEQ ID NO:15 (V_{H} 4.13.1) and SEQ ID NO:21 (V_{L} 4.13.1), respectively; and the amino acid sequences set forth in SEQ ID NO:27 (V_{H} 11.2.1) and SEQ ID NO:33 (V_{L} 11.2.1), respectively.

In yet another embodiment, the amino acid sequence of the heavy chain comprises the amino acid sequence encoded by a nucleic acid comprising the nucleic acid sequences set forth in SEQ ID NOs:1, 13, and 25. In yet another embodiment, the light chain comprises the amino acid sequence encoded by a nucleic acid comprising the nucleic acid sequences set forth in SEQ ID NOs:7, 19 and 31. More preferably, the heavy and light chains comprise the amino acid sequences encoded by nucleic acids comprising the nucleic acid sequences set forth in SEQ ID NO:1 (heavy chain 4.1.1) and SEQ ID NO:7 (light chain 4.1.1), respectively; the nucleic acid sequences set forth in SEQ ID NO:13 (heavy chain 4.13.1) and SEQ ID NO:19 (light chain 4.13.1), respectively; and the nucleic acid sequences set forth in SEQ ID NO:25 (heavy chain 11.2.1) and SEQ ID NO:31 (light chain 11.2.1), respectively.

Furthermore, the antibody can comprise a heavy chain amino acid sequence comprising human CDR amino acid sequences derived from the V_{H} 3-30 or 3-33 gene, or conservative substitutions or somatic mutations therein. The antibody can also comprise CDR regions in its light chain derived from the A27 or O12 gene, i.e., fewer than five, or fewer than ten such mutations. The antibody can also comprise framework regions from those genes, including those that differ by fewer than five, or fewer than ten amino acids. Also included are antibodies with framework regions described herein that have been mutated to reflect the original germ-line sequence.

In other embodiments of the invention, the antibody inhibits binding between CTLA-4 and B7-1, B7-2, or both. Preferably, the antibody can inhibit binding with B7-1 with an IC₅₀ of about 100 nM or lower, more preferably, about 10 nM or lower, for example about 5 nM or lower, yet more preferably, about 2 nM or lower, or even more preferably, for example, about 1 nM or lower. Likewise, the antibody can inhibit binding with B7-2 with an IC₅₀ of about 100 nM or lower, more preferably, 10 nM or lower, for example, even more preferably, about 5 nM or lower, yet more preferably, about 2 nM or lower, or even more preferably, about 1 nM or lower.

Further, in another embodiment, the anti-CTLA-4 antibody has a binding affinity for CTLA-4 of about 10⁻⁸, or greater affinity, more preferably, about 10⁻⁹ or greater affinity, more preferably, about 10⁻¹⁰ or greater affinity, and even more preferably, about 10⁻¹¹ or greater affinity.

The anti-CTLA-4 antibody can compete for binding with an antibody having heavy and light chain amino acid sequences of an antibody selected from the group consisting of 4.1.1, 6.1.1, 11.2.1, 4.13.1 and 4.14.3. Further, the anti-CTLA-4 antibody can compete for binding with an MDX-010 antibody.

In another embodiment, the antibody preferably cross-competes with an antibody having a heavy and light chain sequence, a variable heavy and a variable light chain sequence, and/or the heavy and light CDR sequences of antibody 4.1.1, 4.13.1, 4.14.3, 6.1.1. or 11.2.1. For example, the antibody can bind to the epitope to which an antibody that has heavy and light chain amino acid sequences, variable sequences and/or CDR sequences, of an antibody selected from the group consisting of 4.1.1, 4.13.1, 4.14.3, 6.1.1, or 11.2.1 binds. In another embodiment, the antibody cross-competes with an antibody having heavy and light chain sequences, or antigen-binding sequences, of MDX-010.

In another embodiment, the invention is practiced using an anti-CTLA-4 antibody that comprises a heavy chain comprising the amino acid sequences of CDR-1, CDR-2, and CDR-3, and a light chain comprising the amino acid sequences of CDR-1, CDR-2, and CDR-3, of an antibody selected from the group consisting of 3.1.1, 4.1.1, 4.8.1, 4.10.2, 4.13.1, 4.14.3, 6.1.1, 11.2.1, 11.6.1, 11.7.1, 12.3.1.1, and 12.9.1.1, or sequences having changes from said CDR sequences selected from the group consisting of conservative changes, wherein the conservative changes are selected from the group consisting of replacement of nonpolar residues by other nonpolar residues, replacement of polar charged residues other polar uncharged residues, replacement of polar charged residues by other polar charged residues, and substitution of structurally similar residues; non-conservative substitutions, wherein the non-conservative substitutions are selected from the group consisting of substitution of polar charged residue for polar uncharged residues and substitution of nonpolar residues for polar residues, additions and deletions.

In a further embodiment of the invention, the antibody contains fewer than 10, 7, 5, or 3 amino acid changes from the germline sequence in the framework or CDR regions. In another embodiment, the antibody contains fewer than 5 amino acid changes in the framework regions and fewer than 10 changes in the CDR regions. In one preferred embodiment, the antibody contains fewer than 3 amino acid changes in the framework regions and fewer than 7 changes in the CDR regions. In a preferred embodiment, the changes in the framework regions are conservative and those in the CDR regions are somatic mutations.

In another embodiment, the antibody has at least 80%, more preferably, at least 85%, even more preferably, at least 90%, yet more preferably, at least 95%, more preferably, at least 99%, sequence identity over the heavy and light chain CDR-1, CDR-2 and CDR-3 sequences with the CDR sequences of antibody 3.1.1, 4.1.1, 4.8.1, 4.10.2, 4.13.1, 4.14.3, 6.1.1, 11.2.1, 11.6.1, 11.7.1, 12.3.1.1, and 12.9.1.1. Even more preferably, the antibody shares 100% sequence identity over the heavy and light chain CDR-1, CDR-2 and CDR-3 with the sequence of antibody 3.1.1, 4.1.1, 4.8.1, 4.10.2, 4.13.1, 4.14.3, 6.1.1, 11.2.1, 11.6.1, 11.7.1, 12.3.1.1, and 12.9.1.1.

In yet another embodiment, the antibody has at least 80%, more preferably, at least 85%, even more preferably, at least 90%, yet more preferably, at least 95%, more preferably, at least 99%, sequence identity over the heavy and light chain variable region sequences with the variable region sequences of antibody 3.1.1, 4.1.1, 4.8.1, 4.10.2, 4.13.1, 4.14.3, 6.1.1, 11.2.1, 11.6.1, 11.7.1, 12.3.1.1, and 12.9.1.1. Even more preferably, the antibody shares 100% sequence identity over the heavy and light chain variable region sequences with the sequences of antibody 3.1.1, 4.1.1, 4.8.1, 4.10.2, 4.13.1, 4.14.3, 6.1.1, 11.2.1, 11.6.1, 11.7.1, 12.3.1.1, and 12.9.1.1.

While the anti-CTLA-4 antibodies discussed previously herein may be preferred, the skilled artisan, based upon the disclosure provided herein, would appreciate that the invention encompasses a wide variety of anti-CTLA-4 antibodies and is not limited to these particular antibodies. More particularly, while human antibodies are preferred, the invention is in no way limited to human antibodies; rather, the invention encompasses useful antibodies regardless of species origin, and includes, among others, chimeric humanized and/or primatized antibodies. Also, although the antibodies exemplified herein were obtained using a transgenic mammal, e.g., a mouse comprising a human immune repertoire, the skilled artisan, based upon the disclosure provided herein, would understand that the present invention is not limited to an antibody produced by this or by any other particular method. Instead, the invention includes an anti-CTLA-4 antibody produced by any method, including, but not limited to, a method known in the art (e.g., screening phage display libraries, and the like) or to be developed in the future for producing an anti-CTLA-4 antibody of the invention. Based upon the extensive disclosure provided herein and in, e.g., U.S. Pat. No. 6,682,736, to Bedian et al., and U.S. Pat. App. Pub. No. 2002/0088014, one skilled in the art can readily produce and identify an antibody useful for treatment of breast cancer in combination with a therapeutic agent using the novel methods disclosed herein.

The present invention encompasses human antibodies produced using a transgenic non-human mammal, i.e., XenoMouse™ (Abgenix, Inc., Fremont, Calif.) as disclosed in the U.S. Pat. No. 6,682,736, to Hanson et al.

Another transgenic mouse system for production of “human” antibodies is referred to as “HuMAb-Mouse™” (Medarex, Princeton, N.J.), which contain human immunoglobulin gene miniloci that encodes unrearranged human heavy (mu and gamma) and kappa light chain immunoglobulin sequences, together with targeted mutations that inactivate the endogenous mu and kappa chain loci (Lonberg et al. Nature 368:856-859 (1994), and U.S. Pat. No. 5,770,429).

However, the invention uses human anti-CTLA-4 antibodies produced using any transgenic mammal such as, but not limited to, the Kirin TC Mouse™ (Kirin Beer Kabushiki Kaisha, Tokyo, Japan) as described in, e.g., Tomizuka et al., Proc Natl Acad Sci USA 97:722 (2000); Kuroiwa et al., Nature Biotechnol 18:1086 (2000); U.S. Patent Application Publication No. 2004/0120948, to Mikayama et al.; and the HuMAb-Mouse™ (Medarex, Princeton, N.J.) and XenoMouse™ (Abgenix, Inc., Fremont, Calif.), supra. Thus, the invention encompasses using an anti-CTLA-4 antibody produced using any transgenic or other non-human animal.

Moreover, while the preferred method of producing a human anti-CTLA-4 antibody comprises generation of the antibodies using a non-human transgenic mammal comprising a human immune repertoire, the present invention is in no way limited to this approach. Rather, as would be appreciated by one skilled in the art once armed with the disclosure provided herein, the invention encompasses using any method for production of a human, or any other antibody specific for CTLA-4 produced according to any method known in the art or to be developed in the future for production of antibodies that specifically bind an antigen of interest

Human antibodies can be developed by methods that include, but are not limited to, use of phage display antibody libraries. Using these techniques, antibodies can be generated to CTLA-4 expressing cells, CTLA-4 itself, forms of CTLA-4, epitopes or peptides thereof, and expression libraries thereto (see e.g. U.S. Pat. No. 5,703,057), which can thereafter be screened for the activities described above.

In another embodiment, the antibodies employed in methods of the invention are not fully human, but “humanized”. In particular, murine antibodies or antibodies from other species can be “humanized” or “primatized” using techniques well known in the art. See, e.g., Winter and Harris Immunol. Today 14:43-46 (1993), Wright et al. Crit. Reviews in Immunol. 12:125-168 (1992), and U.S. Pat. No. 4,816,567, to Cabilly et al, and Mage and Lamoyi in Monoclonal Antibody Production Techniques and Applications pp. 79-97, Marcel Dekker, Inc., New York, N.Y. (1987).

As will be appreciated based upon the disclosure provided herein, antibodies for use in the invention can be obtained from a transgenic non-human mammal, and hybridomas derived therefrom, but can also be expressed in cell lines other than hybridomas.

Mammalian cell lines available as hosts for expression are well known in the art and include many immortalized cell lines available from the American Type Culture Collection (ATCC), including but not limited to Chinese hamster ovary (CHO) cells, NS0, HeLa cells, baby hamster kidney (BHK) cells, monkey kidney cells (COS), and human hepatocellular carcinoma cells (e.g., Hep G2). Non-mammalian prokaryotic and eukaryotic cells can also be employed, including bacterial, yeast, insect, and plant cells.

Various expression systems can be used as well known in the art, such as, but not limited to, those described in e.g., Sambrook and Russell, Molecular Cloning, A Laboratory Approach, Cold Spring Harbor Press, Cold Spring Harbor, N.Y. (2001), and Ausubel et al., Current Protocols in Molecular Biology, John Wiley & Sons, NY (2002). These expression systems include dihydrofolate reductase (DHFR)-based systems, among many others. The glutamine synthetase system of expression is discussed in whole or part in connection with European Patents Nos. EP 216 846, EP 256 055, and EP 323 997 and European Patent Application 89303964. In one embodiment, the antibody used is made in NS0 cells using a glutamine synthetase system (GS-NS0). In another embodiment, the antibody is made in CHO cells using a DHFR system. Both systems are well-known in the art and are described in, among others, Barnes et al. Biotech & Bioengineering 73:261-270 (2001), and references cited therein.

Site directed mutagenesis of the antibody CH2 domain to eliminate glycosylation may be preferred in order to prevent changes in either the immunogenicity, pharmacokinetic, and/or effector functions resulting from non-human glycosylation. Further, the antibody can be deglycosylated by enzymatic (see, e.g., Thotakura et al. Meth. Enzymol. 138:350 (1987)) and/or chemical methods (see, e.g., Hakimuddin et al., Arch. Biochem. Biophys. 259:52 (1987)).

Further, the invention encompasses using an anti-CTLA-4 antibody comprising an altered glycosylation pattern. The skilled artisan would appreciate, based upon the disclosure provided herein, that an anti-CTLA-4 antibody can be modified to comprise additional, fewer, or different glycosylations sites compared with the naturally-occurring antibody. Such modifications are described in, e.g., U.S. Patent Application Publication Nos. 2003/0207336, and 2003/0157108, and International Patent Publication Nos. WO 01/81405 and 00/24893.

Additionally, the invention comprises using an anti-CTLA-4 antibody regardless of the glycoform, if any, present on the antibody. Moreover, methods for extensively remodeling the glycoform present on a glycoprotein are well-known in the art and include, e.g., those described in International Patent Publication Nos. WO 03/031464, WO 98/58964, and WO 99/22764, and US Patent Application Publication Nos. 2004/0063911, 2004/0132640, 2004/0142856, 2004/0072290, and U.S. Pat. No. 6,602,684 to Umaña et al.

Further, the invention encompasses using an anti-CTLA-4 antibody with any art-known covalent and non-covalent modification, including, but not limited to, linking the polypeptide to one of a variety of nonproteinaceous polymers, e.g., polyethylene glycol, polypropylene glycol, or polyoxyalkylenes, in the manner set forth in, for example, U.S. Patent Application Publication Nos. 2003/0207346 and 2004/0132640, and U.S. Pat. Nos. 4,640,835; 4,496,689; 4,301,144; 4,670,417; 4,791,192; 4,179,337.

Additionally, the invention encompasses using an anti-CTLA-4 antibody, or antigen-binding portion thereof, chimeric protein comprising, e.g., a human serum albumin polypeptide, or fragment thereof. Whether the chimeric protein is produced using recombinant methods by, e.g., cloning of a chimeric nucleic acid encoding the chimeric protein, or by chemical linkage of the two peptide portions, the skilled artisan would understand once armed with the teachings provided herein that such chimeric proteins are well-known in the art and can confer desirable biological properties such as, but not limited to, increased stability and serum half-life to the antibody of the invention and such molecules are therefore included herein.

Antibodies that are generated for use in the invention need not initially possess a particular desired isotype. Rather, the antibody as generated can possess any isotype and can be isotype switched thereafter using conventional techniques. These include direct recombinant techniques (see, e.g., U.S. Pat. No. 4,816,397), and cell-cell fusion techniques (see e.g., U.S. Pat. No. 5,916,771).

The effector function of the antibodies of the invention may be changed by isotype switching to an IgG1, IgG2, IgG3, IgG4, IgD, IgA, IgE, or IgM for various therapeutic uses. Furthermore, dependence on complement for cell killing can be avoided through the use of bispecifics, immunotoxins, or radiolabels, for example.

Therefore, while the preferred antibodies used in the invention are exemplified by antibodies having the amino acid sequences of 3.1.1, 4.1.1, 4.8.1, 4.10.2, 4.13.1, 4.14.3, 6.1.1, 11.2.1, 11.6.1, 11.7.1, 12.3.1.1, 12.9.1.1, and MDX-010, or, e.g., the sequences of the V regions or CDRs thereof, the present invention is not limited in any way to using these, or any other, particular antibodies. The invention encompasses combining administration of any anti-CTLA-4 antibody of the invention with at least one hormonal therapy agent. Preferably, the antibody is 4.1.1, 4.13.1, 11.2.1, and/or MDX-010. However, any anti-CTLA-4 antibody, or antigen-binding portion thereof, as described elsewhere herein, or as known in the art or developed in the future, can be used in a method of the invention. More particularly, humanized chimeric antibodies, anti-CTLA-4 antibodies derived from any species (including single chain antibodies obtained from camelids as described in, e.g., U.S. Pat. Nos. 5,759,808 and 6,765,087, to Casterman and Hamers), as well as any human antibody, can be combined with a therapeutic agent to practice the novel methods disclosed herein.

The invention also encompasses such antibodies as disclosed in, inter alia, International Patent Publication Nos. WO 00/37504 (published Jun. 29, 2000); WO 01/14424 (published Mar. 1, 2001); WO 93/00431 (published Jan. 7, 1993); and WO 00/32231 (published Jun. 8, 2000), among many others.

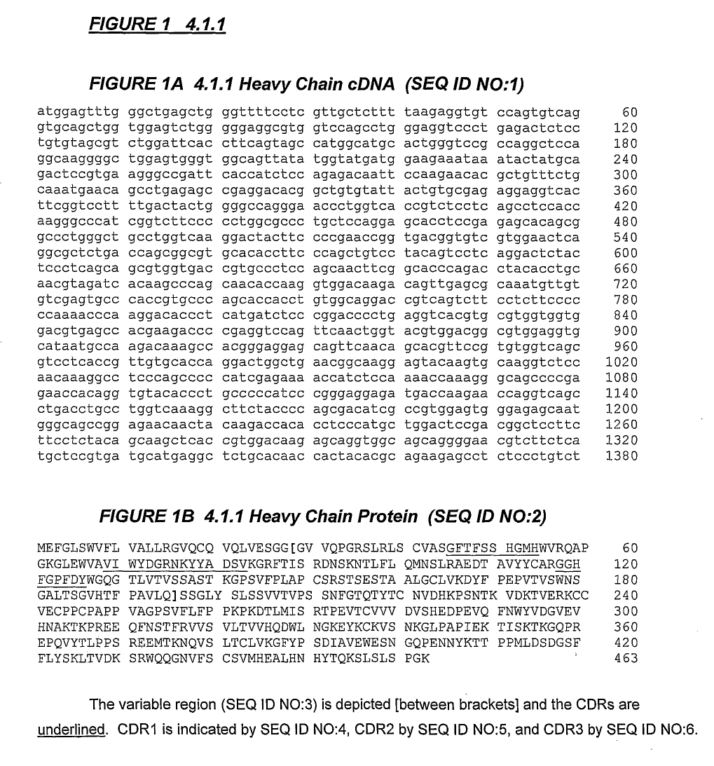

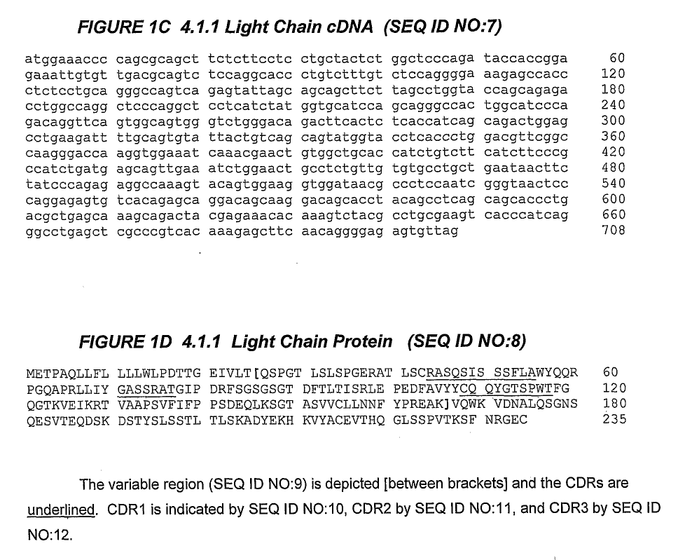

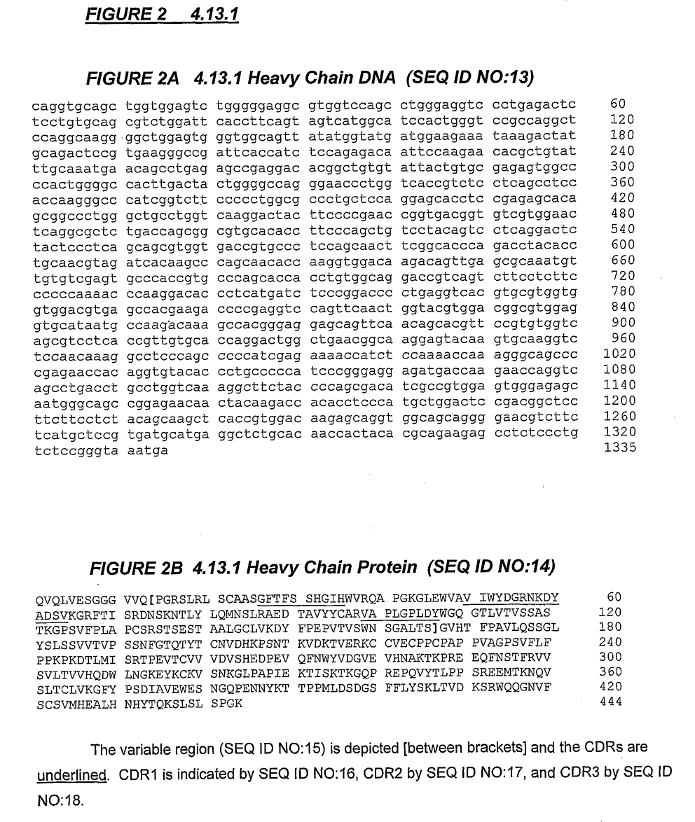

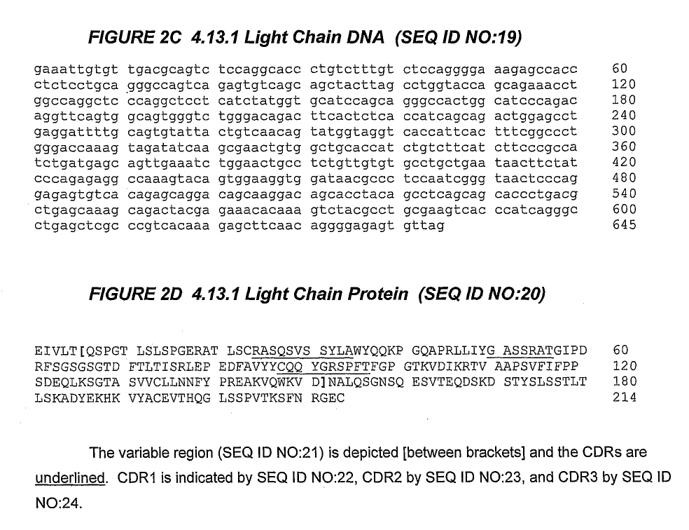

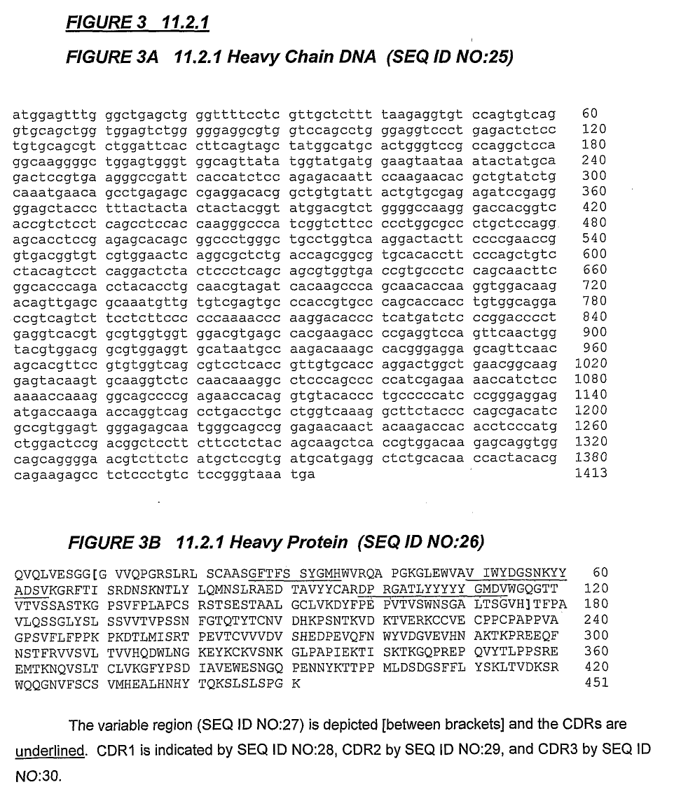

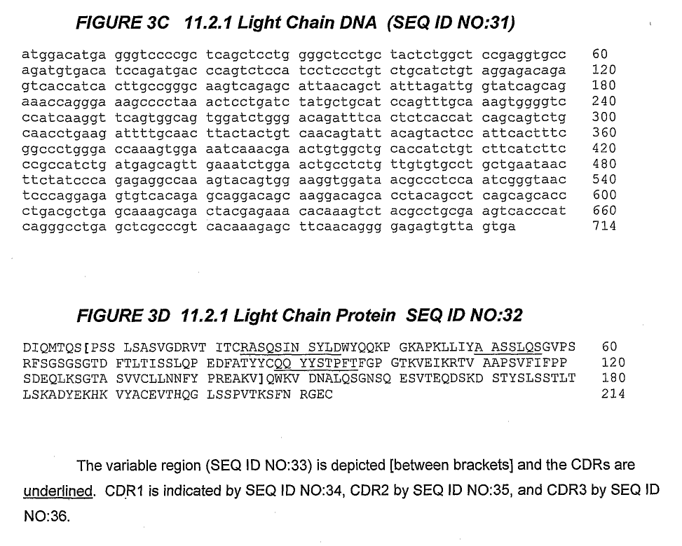

Although antibody 4.1.1, 4.13.1 and 11.2.1 are IgG2 antibodies and the sequences of the variable regions of the antibodies are provided herein (FIGS. 1-3), and in the applications and patents referenced and incorporated herein, it is understood that the full-length sequences of these antibodies are encompassed herein, as well as the use of any antibody comprising the sequences set forth in SEQ ID NOs:1-36, and further comprising any constant region, regardless of isotype as more fully discussed elsewhere herein. Likewise, any antibody comprising the full-length sequence of MDX-010, or any portion thereof, including a sequence encoding an antigen-binding portion of MDX-010, can be administered in combination with at least two hormonal therapy agents thereby treating prostate cancer.

Thus, the skilled artisan, once provided with the teachings provided herein, would readily appreciate that the anti-CTLA-4 antibody-therapeutic agent combination of the invention can comprise a wide plethora of anti-CTLA-4 antibodies.

Further, one skilled in the art, based upon the disclosure provided herein, would understand that the invention is not limited to administration of only a single antibody; rather, the invention encompasses administering at least one anti-CTLA-4 antibody, e.g., 4.1.1, 4.13.1 and 11.2.1, in combination with a therapeutic agent. Moreover, the invention encompasses administering any combination of any known anti-CTLA-4 antibody, including, but not limited to, administering a therapeutic agent in combination with, e.g., 4.1.1, 4.13.1, 11.2.1 and MDX-010. Thus, any combination of anti-CTLA-4 antibodies can be combined with at least one therapeutic agent and the present invention encompasses any such combination and permutation thereof.

### III. CpG ODN

CpG oligonucleotides contain specific sequences found to elicit an immune response. These specific sequences are referred to as “immunostimulatory motifs”, and the oligonucleotides that contain immunostimulatory motifs are referred to as “immunostimulatory oligonucleotide molecules” and equivalently, “immunostimulatory oligonucleotides”. Immunostimulatory oligonucleotides include at least one immunostimulatory motif, and preferably that motif is an internal motif. The term “internal immunostimulatory motif” refers to the position of the motif sequence within an oligonucleotide sequence which is at least one nucleotide longer (at both the 5′ and 3′ ends) than the motif sequence.

CpG oligonucleotides include at least one unmethylated CpG dinucleotide. An oligonucleotide containing at least one unmethylated CpG dinucleotide is a oligonucleotide molecule which contains a cytosine-guanine dinucleotide sequence (i.e., “CpG DNA” or DNA containing a 5′ cytosine linked by a phosphate bond to a 3′ guanine) and activates the immune system. The entire CpG oligonucleotide can be unmethylated or portions may be unmethylated but at least the C of the 5′ CG 3′ must be unmethylated.

The B class of CpG oligonucleotides is represented by the formula:

5′X₁CGX₂ 3′

wherein X₁ and X₂ are nucleotides. In some embodiments, X₁ may be adenine, guanine, or thymine and/or X₂ may be cytosine, adenine, or thymine.

The B class of CpG oligonucleotides is also represented by the formula:

5′X₁X₂CGX₃X₄ 3′

wherein X₁, X₂, X₃, and X₄ are nucleotides. X₂ may be adenine, guanine, or thymine. X₃ may be cytosine, adenine, or thymine.

The B class of CpG oligonucleotide includes oligonucleotides represented by at least the formula:

5′N₁X₁X₂CGX₃X₄N₂ 3′

wherein X₁, X₂, X₃, and X₄ are nucleotides and N is any nucleotide and N₁ and N₂ are oligonucleotide sequences composed of from about 0-25 N's each. X₁X₂ may be a dinucleotide selected from the group consisting of: GpT, GpG, GpA, ApA, ApT, ApG, CpT, CpA, CpG, TpA, TpT, and TpG; and X₃X₄ may be a dinucleotide selected from the group consisting of: TpT, ApT, TpG, ApG, CpG, TpC, ApC, CpC, TpA, ApA, and CpA.

The B class of CpG oligonucleotides is disclosed in PCT Published Patent Applications PCT/US95/01570 and PCT/US97/19791, and U.S. Pat. No. 6,194,388 B1 and U.S. Pat. No. 6,239,116 B1, issued Feb. 27, 2001 and May 29, 2001 respectively.

The immunostimulatory oligonucleotide molecules may have a homogeneous backbone (e.g., entirely phosphodiester or entirely phosphorothioate) or a chimeric backbone. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. Since boranophosphonate linkages have been reported to be stabilized relative to phosphodiester linkages, for purposes of the chimeric nature of the backbone, boranophosphonate linkages can be classified either as phosphodiester-like or as stabilized, depending on the context. For example, a chimeric backbone according to the instant invention could, in one embodiment, include at least one phosphodiester (phosphodiester or phosphodiester-like) linkage and at least one boranophosphonate (stabilized) linkage. In another embodiment, a chimeric backbone according to the instant invention could include boranophosphonate (phosphodiester or phosphodiester-like) and phosphorothioate (stabilized) linkages. A “stabilized internucleotide linkage” shall mean an internucleotide linkage that is relatively resistant to in vivo degradation (e.g., via an exo- or endo-nuclease), compared to a phosphodiester internucleotide linkage. Preferred stabilized internucleotide linkages include, without limitation, phosphorothioate, phosphorodithioate, methylphosphonate and methylphosphorothioate. Other stabilized internucleotide linkages include, without limitation, peptide, alkyl, dephospho type linkages, and others as described above.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated), e.g., as described in U.S. Pat. No. 5,023,243 and European Patent No. 092,574, can be prepared by automated solid phase synthesis using commercially available reagents. Methods for making other DNA backbone modifications and substitutions have been described. Uhlmann E et al. (1990) Chem Rev 90:544; Goodchild J (1990) Bioconjugate Chem 1:165. Methods for preparing chimeric oligonucleotides are also known. For instance patents issued to Uhlmann et al. have described such techniques.

Mixed backbone modified ODN may be synthesized using a commercially available DNA synthesizer and standard phosphoramidite chemistry. (F. E. Eckstein, “Oligonucleotides and Analogues—A Practical Approach” IRL Press, Oxford, UK, 1991, and M. D. Matteucci and M. H. Caruthers, Tetrahedron Lett. 21, 719 (1980)) After coupling, PS linkages are introduced by sulfurization using the Beaucage reagent (R. P. Iyer, W. Egan, J. B. Regan and S. L. Beaucage, J. Am. Chem. Soc. 112, 1253 (1990)) (0.075 M in acetonitrile) or phenyl acetyl disulfide (PADS) followed by capping with acetic anhydride, 2,6-lutidine in tetrahydrofurane (1:1:8; v:v:v) and N-methylimidazole (16% in tetrahydrofurane). This capping step is performed after the sulfurization reaction to minimize formation of undesired phosphodiester (PO) linkages at positions where a phosphorothioate linkage should be located. In the case of the introduction of a phosphodiester linkage, e.g. at a CpG dinucleotide, the intermediate phosphorous-III is oxidized by treatment with a solution of iodine in water/pyridine. After cleavage from the solid support and final deprotection by treatment with concentrated ammonia (15 hrs at 50° C.), the ODN are analyzed by HPLC on a Gen-Pak Fax column (Millipore-Waters) using a NaCl-gradient (e.g. buffer A: 10 mM NaH₂PO₄ in acetonitrile/water=1:4/v:v pH 6.8; buffer B: 10 mM NaH₂PO₄, 1.5 M NaCl in acetonitrile/water=1:4/v:v; 5 to 60% B in 30 minutes at 1 ml/min) or by capillary gel electrophoresis. The ODN can be purified by HPLC or by FPLC on a Source High Performance column (Amersham Pharmacia). HPLC-homogeneous fractions are combined and desalted via a C18 column or by ultrafiltration. The ODN was analyzed by MALDI-TOF mass spectrometry to confirm the calculated mass.

The oligonucleotides of the invention can also include other modifications. These include nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Oligonucleotides which contain diol, such as tetraethyleneglycol or hexaethyleneglycol, at either or both termini have also been shown to be substantially resistant to nuclease degradation.

In some embodiments the oligonucleotides may be soft or semi-soft oligonucleotides. A soft oligonucleotide is an immunostimulatory oligonucleotide having a partially stabilized backbone, in which phosphodiester or phosphodiester-like internucleotide linkages occur only within and immediately adjacent to at least one internal pyrimidine-purine dinucleotide (YZ). Preferably YZ is YG, a pyrimidine-guanosine (YG) dinucleotide. The at least one internal YZ dinucleotide itself has a phosphodiester or phosphodiester-like internucleotide linkage. A phosphodiester or phosphodiester-like internucleotide linkage occurring immediately adjacent to the at least one internal YZ dinucleotide can be 5′, 3′, or both 5′ and 3′ to the at least one internal YZ dinucleotide.

In particular, phosphodiester or phosphodiester-like internucleotide linkages involve “internal dinucleotides”. An internal dinucleotide in general shall mean any pair of adjacent nucleotides connected by an internucleotide linkage, in which neither nucleotide in the pair of nucleotides is a terminal nucleotide, i.e., neither nucleotide in the pair of nucleotides is a nucleotide defining the 5′ or 3′ end of the oligonucleotide. Thus a linear oligonucleotide that is n nucleotides long has a total of n−1 dinucleotides and only n−3 internal dinucleotides. Each internucleotide linkage in an internal dinucleotide is an internal internucleotide linkage. Thus a linear oligonucleotide that is n nucleotides long has a total of n−1 internucleotide linkages and only n−3 internal internucleotide linkages. The strategically placed phosphodiester or phosphodiester-like internucleotide linkages, therefore, refer to phosphodiester or phosphodiester-like internucleotide linkages positioned between any pair of nucleotides in the oligonucleotide sequence. In some embodiments the phosphodiester or phosphodiester-like internucleotide linkages are not positioned between either pair of nucleotides closest to the 5′ or 3′ end.

Preferably a phosphodiester or phosphodiester-like internucleotide linkage occurring immediately adjacent to the at least one internal YZ dinucleotide is itself an internal internucleotide linkage. Thus for a sequence N₁ YZ N₂, wherein N₁ and N₂ are each, independent of the other, any single nucleotide, the YZ dinucleotide has a phosphodiester or phosphodiester-like internucleotide linkage, and in addition (a) N₁ and Y are linked by a phosphodiester or phosphodiester-like internucleotide linkage when N₁ is an internal nucleotide, (b) Z and N₂ are linked by a phosphodiester or phosphodiester-like internucleotide linkage when N₂ is an internal nucleotide, or (c) N₁ and Y are linked by a phosphodiester or phosphodiester-like internucleotide linkage when N₁ is an internal nucleotide and Z and N₂ are linked by a phosphodiester or phosphodiester-like internucleotide linkage when N₂ is an internal nucleotide.

Soft oligonucleotides according to the instant invention are believed to be relatively susceptible to nuclease cleavage compared to completely stabilized oligonucleotides. Without intending to be bound to a particular theory or mechanism, it is believed that soft oligonucleotides of the invention are susceptible to cleavable resulting in fragments with reduced or no immunostimulatory activity relative to full-length soft oligonucleotides. Incorporation of at least one nuclease-sensitive internucleotide linkage, particularly near the middle of the oligonucleotide, is believed to provide an “off switch” which alters the pharmacokinetics of the oligonucleotide so as to reduce the duration of maximal immunostimulatory activity of the oligonucleotide. This can be of particular value in tissues and in clinical applications in which it is desirable to avoid injury related to chronic local inflammation or immunostimulation, e.g., the kidney.

A semi-soft oligonucleotide is an immunostimulatory oligonucleotide having a partially stabilized backbone, in which phosphodiester or phosphodiester-like internucleotide linkages occur only within at least one internal pyrimidine-purine (YZ) dinucleotide. Semi-soft oligonucleotides generally possess increased immunostimulatory potency relative to corresponding fully stabilized immunostimulatory oligonucleotides. Due to the greater potency of semi-soft oligonucleotides, semi-soft oligonucleotides may be used, in some instances, at lower effective concentrations and have lower effective doses than conventional fully stabilized immunostimulatory oligonucleotides in order to achieve a desired biological effect.

It is believed that the foregoing properties of semi-soft oligonucleotides generally increase with increasing “dose” of phosphodiester or phosphodiester-like internucleotide linkages involving internal YZ dinucleotides. Thus it is believed, for example, that generally for a given oligonucleotide sequence with four internal YZ dinucleotides, an oligonucleotide with four internal phosphodiester or phosphodiester-like YZ internucleotide linkages is more immunostimulatory than an oligonucleotide with three internal phosphodiester or phosphodiester-like YZ internucleotide linkages, which in turn is more immunostimulatory than an oligonucleotide with two internal phosphodiester or phosphodiester-like YZ internucleotide linkages, which in turn is more immunostimulatory than an oligonucleotide with one internal phosphodiester or phosphodiester-like YZ internucleotide linkage. Importantly, inclusion of even one internal phosphodiester or phosphodiester-like YZ internucleotide linkage often can be advantageous over no internal phosphodiester or phosphodiester-like YZ internucleotide linkage. In addition to the number of phosphodiester or phosphodiester-like internucleotide linkages, the position along the length of the oligonucleotide can also affect potency.

The soft and semi-soft oligonucleotides will generally include, in addition to the phosphodiester or phosphodiester-like internucleotide linkages at preferred internal positions, 5′ and 3′ ends that are resistant to degradation. Such degradation-resistant ends can involve any suitable modification that results in an increased resistance against exonuclease digestion over corresponding unmodified ends. For instance, the 5′ and 3′ ends can be stabilized by the inclusion there of at least one phosphate modification of the backbone. In a preferred embodiment, the at least one phosphate modification of the backbone at each end is independently a phosphorothioate, phosphorodithioate, methylphosphonate, or methylphosphorothioate internucleotide linkage. In another embodiment, the degradation-resistant end includes one or more nucleotide units connected by peptide or amide linkages at the 3′ end.

A phosphodiester internucleotide linkage is the type of linkage characteristic of oligonucleotides found in nature. The phosphodiester internucleotide linkage includes a phosphorus atom flanked by two bridging oxygen atoms and bound also by two additional oxygen atoms, one charged and the other uncharged. Phosphodiester internucleotide linkage is particularly preferred when it is important to reduce the tissue half-life of the oligonucleotide.

A phosphodiester-like internucleotide linkage is a phosphorus-containing bridging group that is chemically and/or diastereomerically similar to phosphodiester. Measures of similarity to phosphodiester include susceptibility to nuclease digestion and ability to activate RNase H. Thus, for example phosphodiester, but not phosphorothioate, oligonucleotides are susceptible to nuclease digestion, while both phosphodiester and phosphorothioate oligonucleotides activate RNAse H. In a preferred embodiment the phosphodiester-like internucleotide linkage is boranophosphate (or equivalently, boranophosphonate) linkage. U.S. Pat. No. 5,177,198; U.S. Pat. No. 5,859,231; U.S. Pat. No. 6,160,109; U.S. Pat. No. 6,207,819; Sergueev et al., (1998) J Am Chem Soc 120:9417-27. In another preferred embodiment the phosphodiester-like internucleotide linkage is diastereomerically pure Rp phosphorothioate. It is believed that diastereomerically pure Rp phosphorothioate is more susceptible to nuclease digestion and is better at activating RNAse H than mixed or diastereomerically pure Sp phosphorothioate. Stereoisomers of CpG oligonucleotides are the subject of published PCT application PCT/US99/17100 (WO 00/06588). It is to be noted that for purposes of the instant invention, the term “phosphodiester-like internucleotide linkage” specifically excludes phosphorodithioate and methylphosphonate internucleotide linkages.

As described above the soft and semi-soft oligonucleotides of the invention may have phosphodiester like linkages between C and G. One example of a phosphodiester-like linkage is a phosphorothioate linkage in an Rp conformation. Oligonucleotide p-chirality can have apparently opposite effects on the immune activity of a CpG oligonucleotide, depending upon the time point at which activity is measured. (Krieg et al., 2003, Oligonucleotides, 13(6):491-499.) At an early time point of 40 minutes, the Rₚ but not the S_{P} stereoisomer of phosphorothioate CpG oligonucleotide induces JNK phosphorylation in mouse spleen cells. In contrast, when assayed at a late time point of 44 hr, the S_{P} but not the Rₚ stereoisomer is active in stimulating spleen cell proliferation. This difference in the kinetics and bioactivity of the Rₚ and S_{P} stereoisomers does not result from any difference in cell uptake, but rather most likely is due to two opposing biologic roles of the p-chirality. First, the enhanced activity of the Rp stereoisomer compared to the Sp for stimulating immune cells at early time points indicates that the Rp may be more effective at interacting with the CpG receptor, TLR9, or inducing the downstream signaling pathways. On the other hand, the faster degradation of the Rp PS-oligonucleotides compared to the Sp results in a much shorter duration of signaling, so that the Sp PS-oligonucleotides appear to be more biologically active when tested at later time points.

A surprisingly strong effect is achieved by the p-chirality at the CpG dinucleotide itself. In comparison to a stereo-random CpG oligonucleotide the congener in which the single CpG dinucleotide was linked in Rp was slightly more active, while the congener containing an Sp linkage was nearly inactive for inducing spleen cell proliferation.

Thus the oligonucleotides may be heterogeneous in backbone composition thereby containing any possible combination of polymer units linked together.

The term “oligonucleotide” also encompasses oligonucleotides with substitutions or modifications, such as in the sugars. For example, they include oligonucleotides having backbone sugars that are covalently attached to low molecular weight organic groups other than a hydroxyl group at the 2′ position and other than a phosphate group or hydroxy group at the 5′ position. Thus modified oligonucleotides may include a 2′-O-alkylated ribose group. In addition, modified oligonucleotides may include sugars such as arabinose or 2′-fluoroarabinose instead of ribose.

The immunostimulatory oligonucleotides of the instant invention can encompass various chemical modifications and substitutions, in comparison to natural RNA and DNA, involving a phosphodiester internucleotide bridge, or a β-D-ribose unit. Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke S T et al. (1996) Annu Rev Pharmacol Toxicol 36:107-129; and Hunziker J et al. (1995) Mod Synth Methods 7:331-417. An oligonucleotide according to the invention may have one or more modifications, wherein each modification is located at a particular phosphodiester internucleotide bridge and/or at a particular β-D-ribose unit in comparison to an oligonucleotide of the same sequence which is composed of natural DNA or RNA.

For example, the invention relates to an oligonucleotide which may comprise one or more modifications and wherein each modification is independently selected from:
a) the replacement of a phosphodiester internucleotide bridge located at the 3′ and/or the 5′ end of a nucleotide by a modified internucleotide bridge, and
b) the replacement of phosphodiester bridge located at the 3′ and/or the 5′ end of a nucleotide by a dephospho bridge.
c) the replacement of a sugar phosphate unit from the sugar phosphate backbone by another unit, and
d) the replacement of a β-D-ribose unit by a modified sugar unit.

More detailed examples for the chemical modification of an oligonucleotide are as follows:

A phosphodiester internucleotide bridge located at the 3′ and/or the 5′ end of a nucleotide can be replaced by a modified internucleotide bridge, wherein the modified internucleotide bridge is for example selected from phosphorothioate, phosphorodithioate, NR¹R²-phosphoramidate, boranophosphate, α-hydroxybenzyl phosphonate, phosphate-(C₁-C₂₁)—O-alkyl ester, phosphate-[(C₆-C₁₂)aryl-(C₁-C₂₁)—O-alkyl]ester, (C₁-C₈)alkylphosphonate and/or (C₆-C₁₂)arylphosphonate bridges, (C₇-C₁₂)-□-hydroxymethyl-aryl (e.g., disclosed in WO 95/01363), wherein (C₆-C₁₂)aryl, (C₆-C₂₀)aryl and (C₆-C₁₄)aryl are optionally substituted by halogen, alkyl, alkoxy, nitro, cyano, and where R¹ and R² are, independently of each other, hydrogen, (C₁-C₁₈)-alkyl, (C₆-C₂₀)-aryl, (C₆-C₁₄)-aryl-(C₁-C₈)-alkyl, preferably hydrogen, (C₁-C₈)-alkyl, preferably (C₁-C₄)-alkyl and/or methoxyethyl, or R¹ and R² form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleotide by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

A sugar phosphate unit (i.e., a β-D-ribose and phosphodiester internucleotide bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al. (1989) Oligonucleotides Res 17:6129-41), that is, e.g., the replacement by a morpholino-derivative unit; or to build up a polyamide oligonucleotide (“PNA”; as described for example, in Nielsen P E et al. (1994) Bioconjug Chem 5:3-7), that is, e.g., the replacement by a PNA backbone unit, e.g., by 2-aminoethylglycine.

A β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O—(C₁-C₆)alkyl-ribose, preferably 2′-O—(C₁-C₆)alkyl-ribose is 2′-O-methylribose, 2′-O—(C₂-C₆)alkenyl-ribose, 2′-[O—(C₁-C₆)alkyl-O—(C₁-C₆)alkyl]-ribose, 2′-NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

In some embodiments the sugar is 2′-O-methylribose, particularly for one or both nucleotides linked by a phosphodiester or phosphodiester-like internucleotide linkage.

In particular sequences described herein a set of modified bases is defined. For instance the letter Y is used to refer to a nucleotide containing a cytosine or a modified cytosine. A modified cytosine as used herein is a naturally occurring or non-naturally occurring pyrimidine base analog of cytosine which can replace this base without impairing the immunostimulatory activity of the oligonucleotide. Modified cytosines include but are not limited to 5-substituted cytosines (e.g. 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). Some of the preferred cytosines include 5-methyl-cytosine, 5-fluoro-cytosine, 5-hydroxy-cytosine, 5-hydroxymethyl-cytosine, and N4-ethyl-cytosine. In another embodiment of the invention, the cytosine base is substituted by a universal base (e.g. 3-nitropyrrole, P-base), an aromatic ring system (e.g. fluorobenzene or difluorobenzene) or a hydrogen atom (dSpacer).

The letter Z is used to refer to guanine or a modified guanine base. A modified guanine as used herein is a naturally occurring or non-naturally occurring purine base analog of guanine which can replace this base without impairing the immunostimulatory activity of the oligonucleotide. Modified guanines include but are not limited to 7-deazaguanine, 7-deaza-7-substituted guanine (such as 7-deaza-7-(C2-C6)alkynylguanine), 7-deaza-8-substituted guanine, hypoxanthine, N2-substituted guanines (e.g. N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g. N6-methyl-adenine, 8-oxo-adenine) 8-substituted guanine (e.g. 8-hydroxyguanine and 8-bromoguanine), and 6-thioguanine. In another embodiment of the invention, the guanine base is substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom (dSpacer).

The oligonucleotides may have one or more accessible 5′ ends. It is possible to create modified oligonucleotides having two such 5′ ends. This may be achieved, for instance by attaching two oligonucleotides through a 3′-3′ linkage to generate an oligonucleotide having one or two accessible 5′ ends. The 3′3′-linkage may be a phosphodiester, phosphorothioate or any other modified internucleotide bridge. Methods for accomplishing such linkages are known in the art. For instance, such linkages have been described in Seliger, H.; et al., Oligonucleotide analogs with terminal 3′-3′- and 5′-5′-internucleotidic linkages as antisense inhibitors of viral gene expression, Nucleotides & Nucleotides (1991), 10(1-3), 469-77 and Jiang, et al., Pseudo-cyclic oligonucleotides: in vitro and in vivo properties, Bioorganic & Medicinal Chemistry (1999), 7(12), 2727-2735.

Additionally, 3′3′-linked oligonucleotides where the linkage between the 3′-terminal nucleotides is not a phosphodiester, phosphorothioate or other modified bridge, can be prepared using an additional spacer, such as tri- or tetra-ethyleneglycol phosphate moiety (Durand, M. et al, Triple-helix formation by an oligonucleotide containing one (dA)12 and two (dT)12 sequences bridged by two hexaethylene glycol chains, Biochemistry (1992), 31(38), 9197-204, U.S. Pat. No. 5,658,738, and U.S. Pat. No. 5,668,265). Alternatively, the non-nucleotidic linker may be derived from ethanediol, propanediol, or from an abasic deoxyribose (dSpacer) unit (Fontanel, Marie Laurence et al., Sterical recognition by T4 polynucleotide kinase of non-nucleosidic moieties 5′-attached to oligonucleotides; Oligonucleotides Research (1994), 22(11), 2022-7) using standard phosphoramidite chemistry. The non-nucleotidic linkers can be incorporated once or multiple times, or combined with each other allowing for any desirable distance between the 3′-ends of the two ODNs to be linked.

The oligonucleotides are partially resistant to degradation (e.g., are stabilized). A “stabilized oligonucleotide molecule” shall mean an oligonucleotide that is relatively resistant to in vivo degradation (e.g. via an exo- or endo-nuclease). Oligonucleotide stabilization can be accomplished via backbone modifications. Oligonucleotides having phosphorothioate linkages provide maximal activity and protect the oligonucleotide from degradation by intracellular exo- and endo-nucleases. Other modified oligonucleotides include phosphodiester modified oligonucleotides, combinations of phosphodiester and phosphorothioate oligonucleotide, methylphosphonate, methylphosphorothioate, phosphorodithioate, p-ethoxy, and combinations thereof. Oligonucleotides which contain diol, such as tetraethyleneglycol or hexaethyleneglycol, at either or both termini have also been shown to be substantially resistant to nuclease degradation.

The immunostimulatory oligonucleotides may also contain one or more unusual linkages between the nucleotide or nucleotide-analogous moieties. The usual internucleoside linkage is a 3′5′-linkage. All other linkages are considered to be unusual internucleoside linkages, such as 2′5′-, 5′5′-, 3′3′-, 2′2′-, 2′3′-linkages. The nomenclature 2′ to 5′ is chosen according to the carbon atom of ribose. However, if unnatural sugar moieties are employed, such as ring-expanded sugar analogs (e.g. hexanose, cyclohexene or pyranose) or bi- or tricyclic sugar analogs, then this nomenclature changes according to the nomenclature of the monomer. In 3′-deoxy-β-D-ribopyranose analogs (also called p-DNA), the mononucleotides are e.g. connected via a 4′2′-linkage.

If the oligonucleotide contains one 3′3′-linkage, then this oligonucleotide may have two unlinked 5′-ends. Similarly, if the oligonucleotide contains one 5′5′-linkage, then this oligonucleotide may have two unlinked 3′-ends. The accessibility of unlinked ends of nucleotides may be better accessible by their receptors. Both types of unusual linkages (3′3′- and 5′5′) were described by Ramalho Ortigao et al. (Antisense Research and Development (1992) 2, 129-46), whereby oligonucleotides having a 3′3′-linkage were reported to show enhanced stability towards cleavage by nucleases.

Different types of linkages can also be combined in one molecule which may lead to branching of the oligomer. If one part of the oligonucleotide is connected at the 3′-end via a 3′3′-linkage to a second oligonucleotide part and at the 2′-end via a 2′3′-linkage to a third part of the molecule, this results e.g. in a branched oligonucleotide with three 5′-ends (3′3′-, 2′3′-branched).

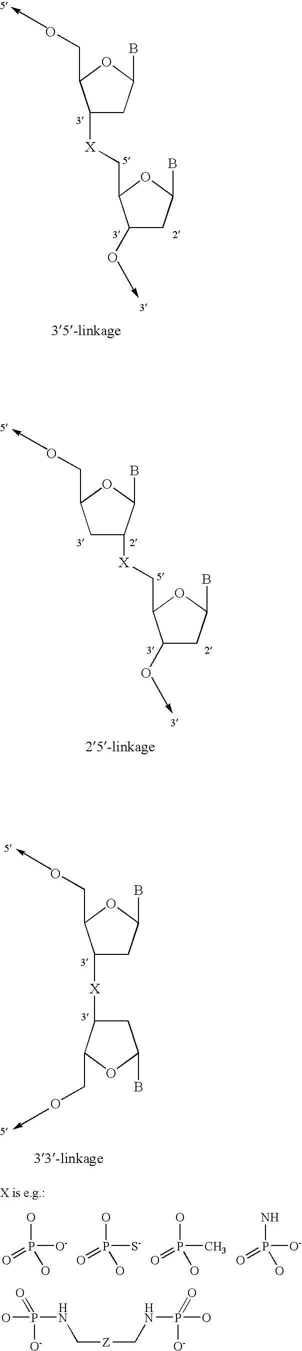

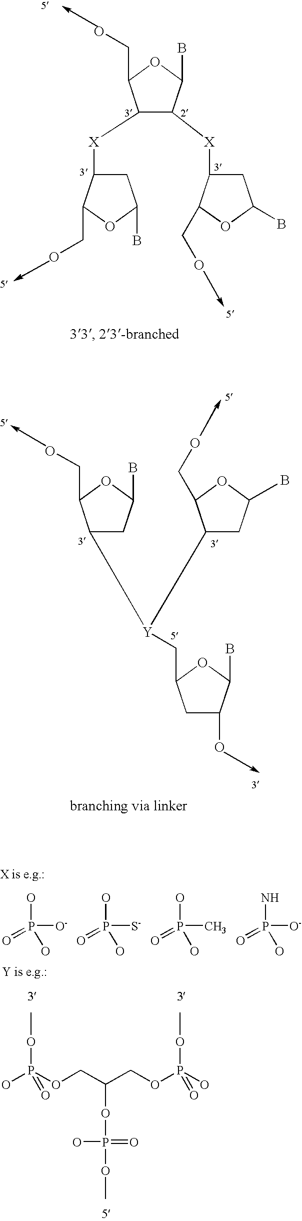

### IV. CpG ODN PF3512676 and anti-CTLA-4 Antibody Combination Therapy

The present invention relates to combination therapy comprising co-administering CpG ODN PF3512676, and an anti-CTLA-4 antibody, preferably, an antibody comprising an antigen-binding portion of antibody 4.1.1, 4.13.1, and 11.2.1, 10D1 (MDX-010), among others. In one embodiment, a combination of an anti-CTLA-4 antibody and a CpG ODN PF3512676 is co-administered to a patient to treat cancer.

Cancer Types

Combination of anti-CTLA-4 antibody and CpG ODN PF3512676 is useful for treatment of primary and secondary (i.e., metastatic) cancers. More specifically, among many potential treatment options, CpG ODN PF3512676 and anti-CTLA-4 combination therapy can be used to treat renal cell carcinoma, breast cancer, colorectal cancer, ovarian cancer, non-small cell lung cancer, melanoma, cutaneous T-cell lymphoma, and NHL (including indolent and aggressive), among many others. While these cancers are preferred, the present invention relates to treatment of a wide variety of malignant cell proliferative disorders, including, but not limited to carcinomas and sarcomas. Further examples include Kaposi's sarcoma, synovial sarcoma, erythroblastoma, mesothelioma, hepatobiliary (hepatic and biliary duct), a primary or secondary brain tumor, lung cancer (NSCLC and SCLC), bone cancer, skin cancer, cancer of the head or neck, cutaneous or intraocular melanoma, bone cancers, cancer of the anal region, stomach cancer, gastrointestinal (gastric, colorectal, and duodenal) cancer, colon cancers, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, prostate cancers, cancer of the penis, testicular cancer, chronic or acute myeloid leukemia, chronic or acute lymphocytic leukemia, lymphocytic lymphomas, cancer of the bladder, cancer of the kidney or ureter, carcinoma of the renal pelvis, pancreatic cancers, neoplasms of the central nervous system (CNS) including primary or secondary CNS tumor, primary CNS lymphoma, spinal axis tumors, brain stem glioma, glioblastoma, meningioma, myoblastoma, astrocytoma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

The cancers to be treated may be refractory cancers. A refractory cancer as used herein is a cancer that is resistant to the ordinary standard of care prescribed. These cancers may appear initially responsive to a treatment (and then recur), or they may be completely non-responsive to the treatment. The ordinary standard of care will vary depending upon the cancer type, and the degree of progression in the subject. It may be a chemotherapy, an immunotherapy, surgery, or radiation, or a combination thereof. Those of ordinary skill in the art are aware of such standards of care. Subjects being treated according to the invention for a refractory cancer therefore may have already been exposed to another treatment for their cancer. Alternatively, if the cancer is likely to be refractory (e.g., given an analysis of the cancer cells or history of the subject), then the subject may not have already been exposed to another treatment.

Examples of refractory cancers include but are not limited to leukemias, melanomas, renal cell carcinomas, colon cancer, liver (hepatic) cancers, pancreatic cancer, Non-Hodgkin's lymphoma, and lung cancer.

Therapy Type

The skilled artisan would appreciate, once provided the teachings disclosed herein, that the invention encompasses CpG ODN therapy combined with an anti-CTLA-4 antibody with, or sequentially (preceding or following) with surgery, radiotherapy, or both, to treat cancer. That is, various treatments can be combined with anti-CTLA-4 antibody-CpG ODN PF3512676 combination therapy, as would be understood by one skilled in the art once armed with the teachings provided herein.

The methods of the invention in certain instances may be useful for replacing existing surgical procedures or drug therapies, although in other instances the present invention is useful in improving the efficacy of existing therapies for treating such conditions. Accordingly combination therapy may be used to treat the subjects that are undergoing or that will undergo a treatment for inter alia cancer. For example, the agents may be administered to a subject in combination with another anti-proliferative (e.g., an anti-cancer) therapy. Suitable anti-cancer therapies include surgical procedures to remove the tumor mass, chemotherapy or localized radiation. The other anti-proliferative therapy may be administered before, concurrent with, or after treatment with the CpG ODN PF3512676/anti-CTLA-4 antibody combination of the invention. There may also be a delay of several hours, days and in some instances weeks between the administration of the different treatments, such that the CpG ODN PF3512676/anti-CTLA-4 antibody combination may be administered before or after the other treatment. The invention further contemplates the use of the CpG ODN PF3512676/anti-CTLA-4 antibody combination in cancer subjects prior to and following surgery, radiation or chemotherapy.

Thus the invention encompasses use of an anti-CTLA-4 antibody in combination with CpG ODN PF3512676 as a neoadjuvant, adjuvant, first line treatment, second-line and/or third-line therapy, in remission induction or maintenance therapy for cancer. That is, in one embodiment, the antibody-CpG ODN PF3512676 combination can be co-administered as neoadjuvant therapy prior to, for instance, surgical resection of a tumor (e.g., prostate, breast and lung cancer). In another embodiment, the antibody-CpG ODN PF3512676 combination can be administered both as a neoadjuvant therapy (i.e., prior to surgery) and also following surgery as an adjuvant therapy. The combination can be used as a first-line treatment instead of another agent (e.g., interferon-alpha).

The methods and compositions of the invention are useful not only in untreated patients but are also useful in the treatment of patients partially or completely unresponsive to other anti-cancer therapies such as but not limited to CpG ODN PF3512676 administered alone (or in combination with another anti-cancer agent) or anti-CTLA-4 antibody administered alone (or in combination with another anti-cancer agent). In various embodiments, the invention provides methods and compositions useful for the treatment of diseases or disorders in patients that have been shown to be or may be refractory or non-responsive to therapies comprising the administration of either or both anti-CTLA-4 antibody and/or CpG ODN PF3512676, and wherein treatment is improved by an enhanced immune response. In one embodiment, the method comprises combining an CpG ODN PF3512676 and an anti-CTLA-4 antibody (preferably, antibody 4.1.1, antibody 4.13.1, antibody 11.2.1, antibody MDX-010, or any combination thereof).

Thus, for example, the combination can be used to treat metastatic renal cell carcinoma as a second-line therapy in cytokine-refractory patients, as a second-line therapy in indolent NHL in further combination with rituximab, and as second-line therapy in CHOP-R (cyclophosphamide, doxorubicin, vincristine, and prednisone, with rituximab) in aggressive NHL, among many others. Combinations of these therapies, where the anti-CTLA-4 antibody-CpG ODN PF3512676 combination is co-administered, are also encompassed in the present invention, such as, but not limited to, where the combination is used for neoadjuvant, adjuvant, first-line, second-line, and third-line therapy, or any combination thereof.

CpG ODN PF3512676 may be used together with an anti-CTLA-4 antibody/(as described above) for remission induction, followed by CpG ODN PF3512676 alone for maintenance therapy. Thus, remission induction therapy may require one or more repeated cycles of combination CpG ODN PF3512676/anti-CTLA-4 antibody therapy. However, once a remission is observed (as will be apparent to a medical practitioner), the subject may be placed on maintenance therapy. Such maintenance therapy may involve monotherapy with CpG ODN PF3512676. For the purpose of maintenance therapy, CpG ODN PF3512676 may be administered once or twice weekly or biweekly, preferably subcutaneously.

While the present invention is exemplified by methods relating to adjuvant, first-line, second-line and/or third-line therapy comprising administering a combination comprising co-administration of an CpG ODN PF3512676 and an anti-CTLA-4 antibody, the skilled artisan, armed with the teachings provided herein, would understand that the invention is not limited to any particular therapy. Rather, methods comprising combined CpG ODN PF3512676 and anti-CTLA-4 antibody therapy encompass use of the combination along the entire disease and treatment continuum. More specifically, the novel methods disclosed herein can provide a therapeutic benefit before and after metastasis, as well as to patients that have become refractory to a chemotherapeutic agent, in that the antibody can enhance an immune response, including any response mediated by therapy as well as any response mediated by CpG ODN PF3512676.

Thus, the present invention is not limited to use of the combinations of the invention solely for neoadjuvant therapy; instead, the invention includes the entire treatment spectrum, including, but not limited to, adjuvant, first-line, second-line and/or third-line therapy for cancer. This is because the data disclosed herein suggest that immunotherapy comprising an anti-CTLA-4 antibody can provide a therapeutic benefit either alone or combined with at least one additional agent, at any point during treatment. That is, the efficacy of a method that mediates release of tumor-specific antigens, such as cytotoxic therapies (e.g., radiation, chemotherapeutics, and the like), where such antigens are exposed to the immune system, can be enhanced by administration of an anti-CTLA-4 antibody of the invention. Indeed, the data disclosed herein further suggest that a synergistic effect is mediated by combined administration of the antibody with CpG ODN PF3512676 for treatment of cancer, more particularly, prostate, breast, CRC, melanoma, pancreatic, lung, NSCLC, NHL, RCC, among many cancers. Therefore, the present invention provides important novel therapeutics for treatment of cancer whereby the patient's immune system is enhanced to provide an anti-tumor effect.

In another embodiment, CpG ODN PF3512676 and an anti-CTLA-4 antibody combination is co-administered to enhance and/or prolong an immune response to a tumor. This is because there may be an interaction between the anti-tumor effect of CpG ODN PF3512676 as, inter alia, a TLR9 agonist and the anti-CTLA-4 antibody-mediated blockade of CTLA-4/B7 signaling of the invention that leads to more effective anti-tumor effect than either agent alone. Thus, without wishing to be bound by any particular theory, the combination of CpG ODN PF3512676 and anti-CTLA-4 antibody can induce a more robust immunological response within the tumor than expected. Without wishing to be bound by any particular theory, the release of tumor antigen(s) mediated by the anti-tumor effects of CpG ODN PF3512676 mediated by, e.g., activation of B lymphocytes and improved antigen-presenting cell (e.g., DCs) function and other immune enhancing effects mediated by activation of TLR9, can increase the immunotherapeutic effect of an anti-CTLA-4, including reducing or breaking immune tolerance to such antigens. This is likely in that CTLA-4 blockade using an antibody and immune activation by CpG ODN PF3512676 have been demonstrated to break tolerance (e.g., reverse or prevent energy or tolerization to tumor antigens) thereby rendering the tumor cells more susceptible to immune attack. Conversely, inhibitory effects from regulatory T cells (Treg) that depend in part on CTLA-4 may limit the effectiveness of CpG immunotherapy, so blocking these effects with an anti-CTLA-4 Ab should improve the efficacy of the CpG. Therefore, the combination of CpG ODN PF3512676 with an anti-CTLA-4 antibody can provide a potential additive or synergistic effect thereby providing an important novel therapeutic treatment for cancer.

In one embodiment, the invention provides a compositions and methods of producing or increasing an anti-tumor response using an anti-CTLA-4 antibody-CpG ODN PF3512676 combination, wherein CpG ODN PF3512676 enhances an anti-tumor response by an amount of antibody which is otherwise sub-optimal for inducing the same level of anti-tumor response when used alone. In certain embodiments, when the CpG ODN PF3512676 is not used in conjunction with an antibody to elicit an anti-tumor response, administering CpG ODN PF3512676 alone does not produce or increase the anti-tumor response. In alternate embodiments, both the CpG ODN PF3512676 and the anti-CTLA-4 antibody can elicit an anti-tumor response alone and/or when administered in combination.

In certain embodiments, the CpG ODN PF3512676 may enhance the effects of the anti-CTLA-4 antibody (or vice-versa) in an additive manner. In a preferred embodiment, the CpG ODN PF3512676 enhances the effects of the anti-CTLA-4 antibody (or vice versa) in a synergistic manner. In another embodiment, the anti-CTLA-4 antibody enhances the effect of an CpG ODN PF3512676 in an additive manner. Preferably, the effects are enhanced in a synergistic manner. Thus, in certain embodiments, the invention encompasses methods of disease treatment or prevention that provide better therapeutic profiles than administration of CpG ODN PF3512676 alone and anti-CTLA-4 antibody alone.

Encompassed by the invention are combination therapies that have additive potency or an additive therapeutic effect while reducing or avoiding unwanted or adverse effects. The invention also encompasses synergistic combinations where the therapeutic efficacy is greater than additive, while unwanted or adverse effects are reduced or avoided. In certain embodiments, the methods of the invention permit treatment or prevention of diseases and disorders wherein treatment is improved by an enhanced anti-tumor response using lower and/or less frequent doses of anti-CTLA-4 antibody and/or CpG ODN PF3512676 to reduce the incidence of unwanted or adverse effects caused by the administration of anti-CTLA-4 antibody and/or CpG ODN PF3512676 alone, while maintaining or enhancing efficacy of treatment, preferably increasing patient compliance, improving therapy and/or reducing unwanted or adverse effects.

### V. Additional Combination Therapy

Based upon the disclosure provided herein, including the immune-enhancing effect of administering an anti-CTLA-4 antibody to a patient, and the combined additive or synergistic effect of co-administering such antibody in combination with CpG ODN PF3512676, it would be appreciated by the skilled artisan that the invention encompasses numerous combination therapies wherein the antibody-CpG ODN PF3512676 is administered to the patient in combination with at least one other therapeutic agent thereby providing a therapeutic benefit. Although many such combinations will be readily apparent to one skilled in the art once armed with the teachings provided herein, several combinations are now discussed. However, the present invention is in no way limited to these combinations, which are set forth herein merely for illustrative purposes.

Co-administration of the antibody-CpG ODN PF3512676 with an additional therapeutic agent (combination therapy) encompasses co-administering both the anti-CTLA-4 antibody, CpG ODN PF3512676, and one or more additional therapeutic agents, and also encompasses co-administering two or more separate pharmaceutical compositions, one comprising the anti-CTLA-4 antibody and the other(s) comprising the CpG ODN PF3512676, and other(s) comprising at least one additional therapeutic agent. Further, although co-administration or combination (conjoint) therapy generally mean that the antibody, CpG ODN PF3512676, and additional therapeutic agents are administered at the same time as one another, it also encompasses simultaneous, sequential or separate dosing of the individual components of the treatment. Additionally, where an antibody is administered intravenously and the anti-cancer agent is administered orally (e.g., chemotherapeutic agent), or by subcutaneous or intramuscular injection, it is understood that the combination is preferably administered as two, three, or more separate pharmaceutical compositions.

When a mammal is subjected to additional chemotherapy, chemotherapeutic agents well-known in the art can be used in combination with an anti-CTLA-4 and CpG ODN PF3512676. Additionally, growth factor inhibitors, biological response modifiers, alkylating agents, intercalating antibiotics, vinca alkaloids, taxanes, selective estrogen receptor modulators (SERMs), angiogenesis inhibitors, among many therapeutic agents, some of which are described below, can be used.

Angiogenesis Inhibitors

Use of an angiogenesis inhibitor in combination with an anti-CTLA-4 antibody has been discussed previously elsewhere herein. Moreover, an angiogenesis inhibitor includes, but is not limited to, bevacizumab (AVASTIN; Genentech), a humanized antibody to VEGF. It can be used in combination with 5FU, and is indicated as a first-line treatment of patients with metastatic carcinoma of the colon or rectum. Agents that directly target angiogenic factors or their receptors offer the prospect for greater activity in receptor-competent hematologic malignancies by interrupting autocrine receptor signaling. Bevacizumab produces sustained neutralization of circulating VEGF and may be useful for treatment of myelodysplastic syndrome (MDS), lymphoma, acute myeloid leukemia (AML), and solid tumors. Receptor tyrosine kinases (RTKIs), including PTK787/ZK222584 (Novartis), are being assessed to treat AML and other receptor-competent hematologic malignancies. The invention also includes treatment of cancer, e.g., renal carcinoma, breast cancer, Non-Hodgkin's lymphoma, colorectal carcinoma, and the like, using a combination of an anti-CTLA-4 antibody and CpG ODN PF3512676, and at least one additional angiogenesis inhibitor, as such inhibitors are well-known in the art or may developed in the future.

Thus, anti-angiogenesis agents, such as MMP-2 (matrix-metalloproteinase 2) inhibitors, MMP-9 (matrix-metalloproteinase 9) inhibitors, and COX-II (cyclooxygenase II) inhibitors, can be used in conjunction with the antibody-CpG ODN PF3512676 combination of the invention. Examples of useful COX-II inhibitors include CELEBREX™ (celecoxib), valdecoxib, rofecoxib, parecoxib, deracoxib, SD-8381, ABT-963, etoricoxib, lumiracoxib, BMS-347070, NS-398, RS 57067, meloxicam. Examples of useful matrix metalloproteinase inhibitors are described in International Patent Publication Nos. WO 96/33172; WO 96/27583; WO 98/07697, WO 98/03516, WO 98/34918, WO 98/34915, WO 98/33768, WO 98/30566, WO 90/05719, WO 99/52910, WO 99/52889, WO 99/29667, European Patent Application Nos. 780386 (published Jun. 25, 1997), 97304971.1 (filed Jul. 8, 1997), 99308617.2 (filed Oct. 29, 1999), 606046 (published Jul. 13, 1994), 931788 (published Jul. 28, 1999), 99302232.1 (filed Mar. 25, 1999), International Application PCT/IB98/01113 (filed Jul. 21, 1998), Great Britain patent application number 9912961.1 (filed Jun. 3, 1999), U.S. Provisional Patent Application No. 60/148,464 (filed Aug. 12, 1999), and U.S. Pat. Nos. 5,863,949, and 5,861,510.

Preferred MMP-2 and MMP-9 inhibitors are those that have little or no activity inhibiting MMP-1. More preferred are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Signal Transduction Inhibitor

The treatments described herein can also be used with signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN (Genentech, Inc., San Francisco, Calif.).

EGFR inhibitors are described in, for example in International Patent Publication Nos. WO 95/19970, WO 98/14451, WO 98/02434, and U.S. Pat. No. 5,747,498, and such substances can be used in the present invention as described herein. EGFR-inhibiting agents include, but are not limited to, the monoclonal antibodies C225, anti-EGFR 22Mab (ImClone Systems Inc., New York, N.Y.), and ABX-EGF (Abgenix Inc., remont, CA), the compounds ZD-1839 (AstraZeneca), BIBX-1382 (Boehringer Ingelheim), MDX-447 (Medarex, Inc., Annandale, N.J.), and OLX-103 (Merck & Co., Whitehouse Station, N.J.), VRCTC-310 (Ventech Research) and EGF fusion toxin (Seragen Inc., Hopkinton, Mass.). These and other EGFR-inhibiting agents can be used in the present invention.

Compounds directed at inhibition of epidermal growth factor receptor (EGFR) tyrosine kinase (TK) represent a relatively new class of antineoplastic drugs that are useful in the method of the present invention. Many human cancers express members of the EGFR family on the cell surface. When a ligand binds to EGFR, it sets off a cascade of cellular reactions that result in increased cell division and influence other aspects of cancer development and progression, including angiogenesis, metastatic spread, and inhibition of apoptosis. EGFR-TK inhibitors may selectively target one of the members of the EGFR family (EGFR (also known as HER1 or ErbB-1), HER2/neu (also known as ErbB-2), HER3 (also known as ErbB-3), or HER4 (also known as ErbB-4)), or may target two or more of them. EGFR-TK inhibitors suitable for use in the present invention include gefitinib (IRESSA), erlotinib (TARCEVA), CI-1033 (Pfizer), GW2016 (GlaxoSmithKline), EKB-569 (Wyeth), PKI-166 (Novartis), CP-724,714 (Pfizer), and BIBX-1382 (Boeringer-Ingelheim). Additional EGFR-TK inhibitors are described in U.S. patent application Ser. No. 09/883,752, filed Jun. 18, 2001.

VEGF inhibitors, in addition to SU11248 (Sugen Inc., San Francisco, Calif.), can also be employed in combination with the antibody and CpG ODN PF3512676 combination. VEGF inhibitors are described for example in International Patent Application No. PCT/IB99/00797 (filed May 3, 1999), International Patent Publication Nos. WO 99/24440; WO 95/21613; WO 99/61422; WO 98/50356; WO 99/10349; WO 97/32856; WO 97/22596; WO 98/54093; WO 98/02438; WO 99/16755; WO 98/02437; U.S. Pat. Nos. 5,834,504; 5,883,113; 5,886,020; and 5,792,783. Other examples of some specific VEGF inhibitors useful in the present invention are IM862 (Cytran Inc., Kirkland, Wash.); IMC-1C11 Imclone antibody, anti-VEGF monoclonal antibody of Genentech, Inc., San Francisco, Calif.; and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc., Woodlands, Tex.) and 2B-1 (Chiron), can furthermore be combined with the antibody-CpG ODN PF3512676 combination, for example those indicated in International Patent Publication Nos. WO 98/02434; WO 99/35146; WO 99/35132; WO 98/02437; WO 97/13760; WO 95/19970; U.S. Pat. Nos. 5,587,458, and 5,877,305. ErbB2 receptor inhibitors useful in the present invention are also described in EP1029853 (published Aug. 23, 2000) and in International Patent Publication No. WO 00/44728, (published Aug. 3, 2000). The erbB2 receptor inhibitor compounds and substance described in the aforementioned PCT applications, U.S. patents, and U.S. provisional applications, as well as other compounds and substances that inhibit the erbB2 receptor, can be used with the antibody in accordance with the present invention.

The treatments of the invention also be used with other agents useful in treating abnormal cell growth or cancer, including, but not limited to other agents capable of enhancing antitumor immune responses, such as additional, different, CTLA4 antibodies, and other agents also capable of blocking CTLA4; and anti-proliferative agents such as farnesyl protein transferase inhibitors (e.g., BMS 214662), and αvβ3 inhibitors, such as the αvβ3 antibody VITAXIN, αvβ5 inhibitors, p53 inhibitors, and the like.

Where the antibody of the invention is administered in combination with another immunomodulatory agent, the immunomodulatory agent can be selected for example from the group consisting of a dendritic cell activator, as well as enhancers of antigen presentation, enhancers of T-cell tropism, inhibitors of tumor-related immunosuppressive factors, such as TGF-β (transforming growth factor beta), and IL-10.

IGF-1R Inhibitor

The present invention encompasses methods comprising combination of CpG ODN PF3512676 with immunotherapy (anti-CTLA-4) further combined with additional agents and therapies. That is, the skilled artisan, based upon the disclosure provided herein, would appreciate that CpG ODN PF3512676 therapy and anti-CTLA-4 antibody combination therapy can be further combined with a wide plethora of therapeutic, surgical, radiation, and other therapeutics, to treat a patient. Therapeutic agents are numerous and have been described in, for instance, U.S. Patent Application Publication No. 2004/0005318, No. 200310086930, No. 2002/0086014, and International Publication No. WO 03/086459, all of which are incorporated by reference herein, among many others. Such therapeutic agents include, but are not limited to, topoisomerase I inhibitors; other antibodies (rituximab, trastuzumab, and the like); chemotherapeutic agents such as, but not limited to, imatinib (GLEEVEC, GLIVEC, or STI571; Novartis), sorafenib (BAY 43-9006; Bayer Pharmaceuticals Corp./Onyx Pharmaceuticals), receptor tyrosine kinase inhibitors, selective estrogen receptor modulators (SERMs), taxanes, vinca alkaloids, temozolomide, angiogenesis inhibitors, EGFR inhibitors, VEGF inhibitors, ErbB2 receptor inhibitors, anti-proliferative agents (e.g., farnesyl protein transferase inhibitors, and αvβ3 inhibitors, αvβ5 inhibitors, p53 inhibitors, and the like), immunomodulators, cytokines, tumor vaccines; tumor-specific antigens; dendritic and stem cell therapies; alkylating agents, folate antagonists; pyrimidine antagonists; anthracycline antibiotics; platinum compounds; costimulatory molecules (e.g., CD4, CD25, PD-1, B7-H3, 4-1BB, OX40, ICOS, CD30, HLA-DR, MHCII, and LFA).

Radiotherapy

Radiation therapy can be co-administered with CpG ODN PF3512676/anti-CTLA-4 antibody combination therapy. Radiotherapy is administered in accordance to well-known radiotherapy methods for treatment of breast cancer. The dose and regimen for radiotherapy can be readily determined by one skilled in the art and is based on the stage of the disease, and other factors well-known in the art.

Palliative Agents

The present invention also encompasses the administration of other therapeutic agents in addition to the first and second components, either concurrently with one or more of those components, or sequentially. Such therapeutic agents include analgesics, cancer vaccines, anti-vascular agents, anti-proliferative agents, anti-emetic agents, and anti-diarrheal agents. Preferred anti-emetic agents include ondensetron hydrochloride, granisetron hydrochloride, and metoclopramide. Preferred anti-diarrheal agents include diphenoxylate and atropine (LOMOTIL), loperamide (IMMODIUM), and octreotide (SANDOSTATIN).

Stem Cell-Based Therapy

The antibody-CpG ODN PF3512676 therapy combination disclosed herein can be combined with stem cell transplantation to provide a therapeutic benefit to a patient afflicted with cancer. Stem cell transplantation may be performed according to the methods known in the art. Some such methods are described in Appelbaum in Harrison's Principles of Internal Medicine, Chapter 14, Braunwald et al., Eds., 15^{th} ed., McGraw-Hill Professional (2001), which is hereby incorporated herein by reference. Thus, the methods of the present invention relate to the treatment of cancer in a mammal who has undergone stem cell transplantation, which methods comprise administering to the mammal an amount of a human anti-CTLA-4 antibody in combination with CpG ODN PF3512676, which antibody-CpG ODN PF3512676 therapy combination is effective in treating the cancer in further combination with stem cell transplantation.

Where the method comprises stem cell transplant, the first dose of the antibody-CpG ODN PF3512676 therapy agent combination can be administered after the immune system of the mammal has recovered from transplantation, for example, in the period of from one to 12 months post transplantation. In certain embodiments, the first dose is administered in the period of from one to three, or one to four months post transplantation. The patient may undergo stem cell transplantation and preparatory treatment(s).

The invention also relates to a method for the treatment of cancer in a mammal comprising the steps of (i) performing stem cell transplantation in the mammal, and (ii) administering an effective amount of a human anti-CTLA-4 antibody in combination with an effective amount of CpG ODN PF3512676. Preferably, the mammal is a human. Stem cell transplantation may be allogeneic or autologous stem cell transplantation. Further, cell transplantation encompasses adoptive transfer of lymphocytes, either from the same patient and/or from a HLA-matched donor.

Further, the methods of the invention can be combined with radiation therapy and stem cell transplant, and any combination of any of the treatments described herein, known in the art, or to be developed in the future.

As pointed out previously elsewhere herein, where an anti-CTLA-4 antibody is combined with a standard cancer treatment, such as, inter alia, chemotherapeutic regimes, it may be possible to reduce the dose of chemotherapeutic reagent administered (Mokyr, M. et al. Cancer Research 58: 5301-5304 (1998)). This is because combined use of an anti-CTLA-4 antibody and an immune enhancing nucleotide, such as CpG ODN PF3512676 as disclosed herein for treatment of cancer, can mediate cell death, or otherwise provide a synergistic effect between the CTLA-4 blockade and the TLR9 agonistic action of the nucleotide. Without wishing to be bound by any particular theory, tumor cell death mediated by the immune response increased or prolonged by anti-CTLA-4 antibody, CpG ODN PF3512676, or the combination thereof, likely results in increased levels of tumor-specific antigen in the antigen presentation pathway, and the anti-CTLA-4 antibody mediates an increased immune response thereto such that coadministration of CpG ODN PF3512676 with the antibody mediates an additive or synergistic increase in the immune response directed to the tumor antigen. Other combination therapies that can result in synergy with anti-CTLA-4-CpG ODN PF3512676 enhancement of the immune response through cell death release of tumor-specific antigens are radiation, surgery, chemotherapy, and administration of a wide plethora of anti-tumor agents well-known in the art and as exemplified herein, among many others. Each of these protocols, and others described elsewhere herein, creates a source of tumor-specific antigen in the host by tumor cell death which may feed tumor antigen into host antigen presentation pathways. Therefore, the combination therapies disclosed herein can provide an increased source of tumor-specific antigens thereby providing an increased immune response to the tumor which, in turn, provides a therapeutic benefit to the patient.

### VI. Dosage Regimens

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the antibody and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present invention. Further, one skilled in the art would understand, once armed with the teachings provided herein, that a therapeutic benefit, such as, but not limited to, detectable decrease in tumor size and/or metastasis, and increased time to recurrence, among many other parameters, can be assessed by a wide variety of methods known in the art for assessing the efficacy of treatment of cancer, and these methods are encompassed herein, as well as methods to be developed in the future.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the antibody are well-known in the relevant art and would be understood to be encompassed by the skilled artisan once provided the teachings disclosed herein.

ODN Dosing

CpG ODN PF3512676 can be administered according to standard dosing regimens well known in the art. Subject doses of CpG ODN PF3512676 for mucosal or local delivery typically range from about 1 μg to 100 mg per administration, which depending on the application could be given daily, weekly, or monthly and any other amount of time therebetween. More typically mucosal or local doses range from about 100 μg to 50 mg per administration, and most typically from about 1 to 10 mg, with 2-4 administrations being spaced days or weeks apart.

Subject doses of the compounds described herein for parenteral delivery for the purpose of inducing a systemic immune response may be typically 2 to 1,000 times higher than the effective mucosal dose, and more typically 2 to 100 times higher, and most typically 5 to 50 times higher.

Doses of CpG ODN PF3512676 for parenteral (including subcutaneous) delivery for inducing an immune response when CpG ODN PF3512676 is administered in combination with other therapeutic agents, such as the antibodies of the invention, or in specialized delivery vehicles typically range from about 10 μg to 1000 mg per administration, which depending on the application could be given daily, weekly, or monthly and any other amount of time therebetween. More typically parenteral doses for these purposes range from about 1 to 500 mg per administration, and most typically from about 5 to 100 mg, with 2-4 administrations being spaced days or weeks apart. In some embodiments, however, parenteral doses for these purposes may be used in a range of 5 to 10,000 times higher than the typical doses described above.

In some embodiments, the ODN is administered once weekly in amounts ranging from 10-40 mg total. ODN may be administered in doses of 5 or 10 mg each, thereby resulting in multiple boli or injections depending on the total amount to be administered. For example, if the total amount to be administered is 10 mg, this may be administered by for example 2×5 mg injection doses. As another example, if the total amount to be administered is 40 mg, this may be administered by for example 4×10 mg injection doses.

Antibody Dosing

An exemplary, non-limiting range for a therapeutically effective amount of an antibody administered according to the invention is at least about 0.1 mg/kg, at least about 0.3 mg/kg, at least about 1 mg/kg, at least about 5 mg/kg, at least about 6 mg/kg, at least about 10 mg/kg, at least about 15 mg/kg, at least about 20 mg/kg, at least about 30 mg/kg, or at least about 50 mg/kg. For example, a therapeutically effective amount of antibody can range from about 0.1-30 mg/kg, or for example about 0.3-25 mg/kg, or for example about 1-20 mg/kg, or for example about 3-20 mg/kg, or for example about 5-20 mg/kg, or for example about 10-20 mg/kg, or about 3-15 mg/kg, or about 5-15 mg/kg, or about 10-15 mg/kg.

In another embodiment, the antibody is administered at a dose of at least 0.3 mg/kg, preferably, at least 1 mg/kg, more preferably, at least 3 mg/kg, yet more preferably, at least 5 mg/kg, preferably, at least 6 mg/kg, even more preferably, at least 10 mg/kg, yet more preferably, at least 15 mg/kg, and even more preferably, at least 20 mg/kg.

Further, the antibody is administered by i.v. infusion at a dose ranging from about 0.1 mg/kg to 50 mg/kg, more preferably, from about 0.3 mg/kg to 20 mg/kg, more preferably, from about 1 mg/kg to 15 mg/kg, even more preferably from about 3 mg/kg to 15 mg/kg, even more preferably, from about 6 mg/kg to 15 mg/kg. In one embodiment, the antibody is administered in an intravenous formulation as a sterile aqueous solution containing about 5 to 20 mg/ml of antibody, in an appropriate buffer system.

Further, an exemplary dose escalation protocol can be used to determine the maximum tolerated dose (MTD), to assess dose limiting toxicity (DLT), if any, associated with administration of antibody-CpG ODN PF3512676 combination therapy, and the like, comprises administering increasing doses, such as, but not limited to about 0.1 mg/kg, 0.3 mg/kg, 1 mg/kg, 3 mg/kg, 6 mg/kg, 7 mg/kg, 10 mg/kg, 12 mg/kg, 15 mg/kg, or more than 15 mg/kg, or any combination thereof, more preferably, successive doses of 0.1 mg/kg, 0.3 mg/kg, 1 mg/kg, 3 mg/kg, 6 mg/kg, 10 mg/kg, 15 mg/kg or 20 mg/kg are administered and the patient is assessed for toxicity, if any, as well as for efficacy of treatment, among other parameters. Such studies to determine toxicity and efficacy of dose regimens are well-known in the art.

Timing of Administration

CpG ODN PF3512676 may be administered substantially simultaneously or sequentially with anti-CTLA-4 antibodies of the invention. When administration is simultaneous, the ODN and the antibody may be in the same or separate formulations although they are administered at the same time. The term “substantially simultaneously” means that the compounds are administered within minutes of each other (e.g., within 10 minutes of each other) and intends to embrace joint administration as well as consecutive administration, but if the administration is consecutive it is separated in time for only a short period (e.g., the time it would take a medical practitioner to administer two compounds separately). As used herein, concurrent administration and substantially simultaneous administration are used interchangeably. Sequential administration refers to temporally separated administration of the ODN and the antibody. The separation in time between the administration of these compounds are deliberately longer than the time it takes to administer two medicaments separately, one after the other, without intended delay. Co-administration thus encompasses any temporal combination of administration of the antibody and the CpG ODN PF3512676 such that administration of the two mediates a therapeutic benefit to the patient that is detectably greater than administration of either agent in the absence of the other.

The CpG ODN may be administered before, concurrently with, or after (or any combination thereof) administration of the antibody, and vice versa. The CpG ODN may be administered daily (including one or more administrations per day), every other day, every three days, every four days, every five days, every six days, or every week, every month, every two months, every three months, every four months, every five months, every six months, or every year. The antibody may be administered daily, every other day, every three days, every four days, every five days, every six days, every week, every two weeks, monthly, or every twenty days, every 25 days, every 28 days, every 30 days, every 40 days, every 50 days, every two months, every 70 days, every 80 days, every three months, every six months or yearly. A single dose or multiples doses of the antibody may be administered. Alternatively, at least one dose, or at least three, six or 12 doses may be administered. The doses may be administered, for example. The administration of the ODN and antibody may alternate.

In one embodiment, part of the dose is administered by an intravenous bolus and the rest by infusion of the antibody formulation. For example, an intravenous injection of the antibody may be given as a bolus, and the rest of a predetermined antibody dose may be administered by intravenous injection. A predetermined dose of the antibody may be administered, for example, over a period of about an hour and a half to about five hours.

In one embodiment, CpG ODN PF3512676 and the antibody are co-administered in that CpG ODN PF3512676 is administered at the doses recited herein, preferably parenterally (e.g., by subcutaneous or IV route). In another embodiment, the anti-CTLA-4 antibody is administered first to block the inhibitory effects that would limit the efficacy of the CpG ODN. In this embodiment, the anti-CTLA-4 antibody is given preferably from 1 week to 1 day prior to the CpG ODN, and most preferably from 2-3 days prior to the CpG ODN.

In another embodiment, the CpG ODN is given first, to prime the immune system to have a better immune activation response to the anti-CTLA-4 antibody and any other immunotherapies or other therapy that may be given in conjunction with this (e.g., tumor vaccine or etc.). In this embodiment, the CpG ODN is given preferably from 1 week to 1 day prior to the anti-CTLA-4 antibody, and most preferably from 2-3 days prior to the anti-CTLA-4 antibody.

While any suitable resting period can be used between administration of CpG ODN PF3512676 and anti-CTLA-4 antibody, the present invention does not require a waiting period and the antibody and CpG ODN PF3512676 can be co-administered substantially simultaneously. Thus, in one embodiment, the antibody is administered as a single injection and CpG ODN PF3512676 is administered about 1-7 days either before or after the antibody.

The antibody or antibody fragment may be administered with the CpG ODN PF3512676 in a multi-day or multi-week cycle. The multi-day cycle be a 2, 3, 4, 5, 6, 7, 8, 9, 10 or more day cycle, or a 2, 3, 4 or more week cycle. The antibody or fragment thereof may be administered on the first day of such a cycle, followed by administration of the CpG ODN PF3512676 on the first day of each week of a multiweek cycle. For example, the CpG ODN PF3512676 may be administered on days 1, 7 and 14 of a three week cycle. The three week cycle may be repeated once, two three times or more. The entire treatment may be preceded by administration of either the ODN or the antibody alone, for example in order to prime the immune system or render the subject more responsive to the subsequent therapy.

Additional cycles of antibody and CpG ODN PF3512676 can be provided as determined by art-recognized methods. However, the present invention is not limited to these or any particular dosage or administration regimens for administering CpG ODN PF3512676 in combination with an anti-CTLA-4 antibody. Rather, the optimal dose, route and regimen for administration of the antibody and CpG ODN PF3512676 can be readily determined by one of ordinary skill in the relevant art using well-known methods.

The antibody-CpG ODN PF3512676 combination can be administered as a neoadjuvant therapy prior to surgery, radiation therapy, or any other treatment, in order to sensitize the tumor cells or to otherwise confer a therapeutic benefit to the patient. Additionally, the combination can be co-administered as neoadjuvant therapy following localized treatment (e.g., surgery, radiation, or both).

Further, the combination can be administered as a second line therapy, such as, but not limited to, once any first line therapy has failed. Alternatively, the combination can be administered concurrently with first line therapy, and or at any point during first line therapy, which can be administered following initial treatment.

This is because a combination of an anti-CTLA-4 antibody and CpG ODN PF3512676 can provide a therapeutic benefit once first line therapy has failed, once systemic adjuvant therapy has failed, and the like. Thus, the invention encompasses administration of a antibody and CpG ODN PF3512676 in combination, with or without additional therapy, including, but not limited to, hormonal (e.g., anti-androgen, aromatase inhibitor, and the like), radiotherapy, and any additional therapeutic agent (chemotherapy, signal inhibition therapy, among others), and the like, as would be appreciated by one skilled in the art based upon the disclosure provided herein.

### VII. Pharmaceutical Compositions

The invention also relates to an article of manufacture (e.g., dosage form adapted for i.v. administration) comprising a human anti-CTLA-4 antibody in the amount effective to treat cancer (e.g., at least 1 mg/kg, at least 3 mg/kg, at least 5 mg/kg, at least 10 mg/kg, at least 15 mg/kg, or at least 20 mg/kg) and a therapeutically effective amount of CpG ODN PF3512676. In certain embodiments, the article of manufacture comprises a container or containers comprising a human anti-CTLA-4 antibody, CpG ODN PF3512676, and a label and/or instructions for use to treat cancer.

The invention encompasses the preparation and use of pharmaceutical compositions comprising a human anti-CTLA-4 antibody of the invention as an active ingredient in combination with and without CpG ODN PF3512676. Such a pharmaceutical composition may consist of each active ingredient alone, as a combination of at least one active ingredient (e.g., an effective dose of an anti-CTLA-4, an effective dose of CpG ODN PF3512676) in a form suitable for administration to a subject, or the pharmaceutical composition may comprise the active ingredient and one or more pharmaceutically acceptable carriers, one or more additional (active and/or inactive) ingredients, or some combination of these.

CpG ODN PF3512676 may be directly administered to the subject or may be administered in conjunction with a nucleic acid delivery complex. A nucleic acid delivery complex shall mean a nucleic acid molecule associated with (e.g. ionically or covalently bound to; or encapsulated within) a targeting means (e.g. a molecule that results in higher affinity binding to target cell. Examples of nucleic acid delivery complexes include oligonucleotides associated with a sterol (e.g. cholesterol), a lipid (e.g. a cationic lipid, virosome or liposome), or a target cell specific binding agent (e.g. a ligand recognized by target cell specific receptor). Preferred complexes may be sufficiently stable in vivo to prevent significant uncoupling prior to internalization by the target cell. However, the complex can be cleavable under appropriate conditions within the cell so that the nucleic acid is released in a functional form.

Delivery vehicles or delivery devices for delivering antigen and oligonucleotides to surfaces have been described. The CpG ODN PF3512676 and/or the antigen and/or other therapeutics may be administered alone (e.g., in saline or buffer) or using any delivery vehicles known in the art. For instance the following delivery vehicles have been described: Cochleates; Emulsomes, ISCOMs; Liposomes; Live bacterial vectors (e.g., Salmonella, Escherichia coli, Bacillus calmatte-guerin, Shigella, Lactobacillus); Live viral vectors (e.g., Vaccinia, adenovirus, Herpes Simplex); Microspheres; Oligonucleotide vaccines; Polymers; Polymer rings; Proteosomes; Sodium Fluoride; Transgenic plants; Virosomes; Virus-like particles, and cationic lipids, peptides, or other carriers that have a charge interaction with the polyanionic oligonucleotide. Other delivery vehicles are known in the art and some additional examples are provided below in the discussion of vectors.

In one embodiment, the antibody is administered parenterally (e.g., intravenously) in an aqueous solution while the CpG ODN PF3512676 is administered by subcutaneous injection. Preferred formulations and dosage forms of the CpG ODN PF3512676 are described in U.S. Patent Application Publication No. US2004/0198680, the disclosure of which is incorporated herein by reference in its entirety. However, the skilled artisan would understand, based upon the disclosure provided herein, that the invention is not limited to these, or any other, formulations, doses, routes of administration, and the like. Rather, the invention encompasses any formulation or method of administering an antibody in combination with a CpG ODN PF3512676, including, but not limited to, administering each agent separately in a different formulation via a different route of administration (e.g., administering an anti-CTLA-4 antibody i.v., while co-administering an CpG ODN PF3512676 subcutaneously, among many others. Thus, the following discussion describes various formulations for practicing the methods of the invention comprising administration of any anti-CTLA-4 antibody in combination with an CpG ODN PF3512676, but the invention is not limited to these formulations, but comprises any formulation as can be readily determined by one skilled in the art once armed with the teachings provided herein for use in the methods of the invention.

The antibodies employed in the invention can be incorporated into pharmaceutical compositions suitable for administration to a subject. Typically, the pharmaceutical composition comprises the antibody and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, trehalose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the antibody or antibody portion.

The antibodies may be in a variety of forms. These include, for example, liquid, semi solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yields a powder of the active ingredient plus any additional desired ingredient from a previously sterile filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies and/or CpG ODN PF3512676 can be administered by a variety of methods known in the art, including, without limitation, oral, parenteral, mucosal, by-inhalation, topical, buccal, nasal, and rectal. For many therapeutic applications, the preferred route/mode of administration is subcutaneous, intramuscular, intravenous or infusion. Non-needle injection may be employed, if desired. As will be appreciated by the skilled artisan, the route and/or mode of administration will vary depending upon the desired results.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the antibody and the particular therapeutic or prophylactic effect to be achieved, and (b) the limitations inherent in the art of compounding such an active compound for the treatment of sensitivity in individuals.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In one embodiment, the antibody is administered in an intravenous formulation as a sterile aqueous solution containing 5 or 10 mg/ml of antibody, with sodium acetate, polysorbate 80, and sodium chloride at a pH ranging from about 5 to 6. Preferably, the intravenous formulation is a sterile aqueous solution containing 5 or 10 mg/ml of antibody, with 20 mM sodium acetate, 0.2 mg/ml polysorbate 80, and 140 mM sodium chloride at pH 5.5.

In one embodiment, part of the dose is administered by an intravenous bolus and the rest by infusion of the antibody formulation. For example, a 0.01 mg/kg intravenous injection of the antibody may be given as a bolus, and the rest of a predetermined antibody dose may be administered by intravenous injection. A predetermined dose of the antibody may be administered, for example, over a period of an hour and a half to two hours to five hours.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics, anti-diarrheals, chemotherapeutic agents, cytokines, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations as discussed below. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

A composition of the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, (1978). Pharmaceutical compositions are preferably manufactured under GMP conditions.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

The anti-CTLA-4 antibody/CpG ODN PF3512676 active ingredient combination of the invention can be administered to an animal, preferably a human. While the precise dosage administered of each active ingredient will vary depending upon any number of factors, including but not limited to, the type of animal and type of disease state being treated, the age of the animal and the route(s) of administration.

An antibody-CpG ODN PF3512676 combination of the invention may be co-administered with numerous other compounds (antihormonal therapy agents, cytokines, chemotherapeutic and/or antiviral drugs, among many others). Alternatively, the compound(s) may be administered an hour, a day, a week, a month, or even more, in advance of the antibody-CpG ODN PF3512676 combination, or any permutation thereof. Further, the compound(s) may be administered an hour, a day, a week, or even more, after administration of radiation, stem cell transplant, or administration of any therapeutic agent (e.g., cytokine, chemotherapeutic compound, and the like), or any permutation thereof. The frequency and administration regimen will be readily apparent to the skilled artisan and will depend upon any number of factors such as, but not limited to, the type and severity of the disease being treated, the age and health status of the animal, the identity of the compound or compounds being administered, the route of administration of the various compounds, and the like. Several instructive examples demonstrating methods of co-administering an antibody-CpG ODN PF3512676 to treat cancer are provided, but the invention is not limited in any way to these examples, which merely serve to illustrate methods encompassed by the invention.

### VIII. Kits

The invention includes various kits for treatment of cancer, The kits comprise a therapeutically effective amount of a human anti-CTLA-4 antibody of the invention and a therapeutically effective amount of CpG ODN PF3512676, along with an applicator and instructional materials which describe use of the combination to perform the methods of the invention. Although exemplary kits are described below, the contents of other useful kits will be apparent to the skilled artisan in light of the present disclosure. Each of these kits is included within the invention.

The invention includes a kit for treatment of renal cell carcinoma in a patient in need thereof. The kit includes a human anti-CTLA-4 antibody of the invention and CpG ODN PF3512676. The kit further comprises an applicator, including, but not limited to, a syringe, for administration of the components of the kit to a patient. Further, the kit comprises an instructional material setting forth the pertinent information for the use of the kit to treat breast cancer in the patient.

More preferably, the kit comprises at least one anti-CTLA-4 antibody selected from 4.1.1, 4.8.1, 4.10.2, 4.13.1, 4.14.3, 6.1.1, 11.2.1, 11.6.1, 11.7.1., 12.3.1.1, 12.9.1.1, and MDX-010, even more preferably, the antibody is 4.13.1, 11.2.1, and MDX-010.

The invention encompasses a kit comprising any combination of an anti-CTLA-4 antibody and CpG ODN PF3512676. While such kit is preferred, the invention is not limited to this particular combination. Further, the kit can comprise a wide plethora of additional agents for treatment of cancer. Such agents are set forth previously and include chemotherapeutic compounds, cancer vaccines, TLR agonists other than an CpG ODN PF3512676, other CpG ODNs, receptor tyrosine kinase inhibitors (such as, but not limited to, SU11248), agents useful in treating abnormal cell growth or cancer, antibodies or other ligands that inhibit tumor growth by binding to IGF-1R, a chemotherapeutic agent (taxane, vinca alkaloid, platinum compound, intercalating antibiotics, among many others), and cytokines, among many others, as well as palliative agents to treat, e.g., any toxicities that arise during treatment such as, but not limited to, an anti-diarrheal, an anti-emetic, and the like.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

### Anti-CTLA-4 Antibody in Combination with CpG ODN PF3512676 for Treatment of Breast Cancer

Following surgery/radiotherapy, if any, patients having metastatic breast cancer with at least one lesion that can be accurately measured in two dimensions by conventional CT scan or by spiral CT scan are given CpG ODN PF3512676 per established protocols. Briefly, CpG ODN PF3512676 is administered subcutaneously or IV at doses of 0.02 to 20 mg/kg, and most preferably about 0.2 mg/kg for SC and 2 mg/kg for IV.

The patient is further administered a single IV infusion (100 mL/hr) of anti-CTLA-4 antibody 11.2.1 as described herein at a dose of about 10 mg/kg, given between 7 days prior or 7 days after the CpG ODN PF3512676 treatment. The antibody treatment is repeated after 28 days without escalation of the anti-CTLA-4 antibody dose, every 28 days thereafter for maximum of 12 cycles in the absence of intolerable toxicity or disease progression.

The patient can be premedicated with antihistamine (H1) at least one half hour prior to infusion of anti-CTLA-4. However, although pre-medication can be administered, preferably, the patient is not typically pretreated. More preferably, administration of antihistamine (H1), and/or other therapeutic measures, are provided to patients who experience infusion reactions.

Anti-emetics and anti-diarrheals, among other palliative treatments, are given as appropriate during and after treatment.

CpG ODN PF3512676 is administered sequentially or simultaneously with human anti-CTLA-4 antibody 11.2.1, either once, or repeatedly, as determined.

The anti-CTLA-4 antibody is provided in 10 ml clear glass vials with a rubber stopper and an aluminum seal. Each vial contains 5 mg/ml (with a nominal fill of 50 mg/vial) of anti-CTLA-4 antibody, in a sterile aqueous solution comprising 20 mM sodium acetate, 0.2 mg/ml polysorbate 80, and 140 mM sodium chloride at pH 5.5.

CpG ODN PF3512676 is provided in a pharmaceutically acceptable sterile preservative-free phosphate buffered saline solution at various concentrations for parenteral administration.

For all patients, ECOG performance status, vital signs, and body weight are assessed pre-dose, and vital signs can be repeated post-dose, as clinically indicated. A physical examination (including ophthalmologic assessment and signs of autoimmunity) is performed on Day 1. Samples for hematology panel (hematocrit, RBC count, WBC count, differential), chemistry (Alkaline Phosphatase, calcium, chloride, GGT, LDH, magnesium, phosphorus, random glucose, sodium, urea, uric acid), urinalysis (blood, protein), others (activated partial thromboplastin time [APTT], prothrombin time (PT), autoantibody panel, C reactive protein, TSH, T3, T4, amylase, lipase, serum C3, C4, serum Ig level), are obtained.

Baseline human anti-human antibody (HAHA) titer is determined and pharmacokinetic (PK) specimen is obtained pre-dose.

The following endpoints are measured: PK parameters, HAHA, response rate and time to progression. Time to progression and overall survival are calculated using the Kaplan-Meier product limit method.

### EQUIVALENTS

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

The disclosures of each and every patent, patent application, and publication cited herein are hereby incorporated herein by reference in their entirety.

What is claimed is:

1. A method for the treatment of cancer in a patient in need of such treatment, said method comprising administering to said patient a therapeutically effective amount of an anti-CTLA-4 antibody, or antigen-binding portion thereof, in combination with a therapeutically effective amount of CpG ODN PF3512676.

2. The method of claim 1, wherein said CpG ODN PF3512676 is administered daily, every other day, twice a week, or weekly.

3. The method of claim 1, wherein said treatment is a therapy selected from the group consisting of neoadjuvant therapy, adjuvant therapy, first-line therapy, second-line therapy, and third-line therapy.

4. The method of claim 1, wherein said therapeutically effective amount of said human anti-CTLA-4 antibody ranges from about 0.1 mg/kg to 50 mg/kg.

5. The method of claim 4, wherein said therapeutically effective amount of said human anti-CTLA-4 antibody ranges from about 0.3 mg/kg to 20 mg/kg.

6. The method of claim 5, wherein said therapeutically effective amount of said human anti-CTLA-4 antibody is selected from the group consisting of at least 1 mg/kg, at least 3 mg/kg, at least 6 mg/kg, at least 10 mg/kg, and at least 15 mg/kg.

7. The method of claim 1, wherein said cancer is selected from the group consisting of breast cancer, prostate cancer, ovarian cancer, pancreatic cancer, melanoma, lung cancer, acute myeloid leukemia, colorectal carcinoma, renal cell carcinoma, cutaneous T-cell lymphoma, Non-Hodgkin's lymphoma, gastric cancers, head and neck cancer, liver cancer, cervical cancer, brain cancer, and sarcoma.

8. The method of claim 1, wherein said anti-CTLA-4 antibody, or antigen-binding portion thereof, is at least one antibody selected from the group consisting of:
(a) a human antibody having a binding affinity for CTLA-4 of about 10⁻⁸ or greater, and which inhibits binding between CTLA-4 and B7-1, and binding between CTLA-4 and B7-2;
(b) a human antibody having an amino acid sequence comprising at least one human CDR sequence that corresponds to a CDR sequence from an antibody selected from the group consisting of 4.1.1, 4.8.1, 4.10.2, 4.13.1, 4.14.3, 6.1.1, 11.2.1, 11.6.1, 11.7.1., 12.3.1.1, 12.9.1.1, and 10D1;
(c) a human antibody having the amino acid sequence of heavy and light chains of an antibody selected from the group consisting of 4.1.1, 4.8.1, 4.10.2, 4.13.1, 4.14.3, 6.1.1, 11.2.1, 11.6.1, 11.7.1., 12.3.1.1, 12.9.1.1, and 10D1;
(d) an antibody, or antigen-binding portion thereof, that competes for binding with CTLA-4 with at least one antibody having the amino acid sequence of an antibody selected from the group consisting of 4.1.1, 4.8.1, 4.10.2, 4.13.1, 4.14.3, 6.1.1, 11.2.1, 11.6.1, 11.7.1., 12.3.1.1, 12.9.1.1, and 10D1; and
(e) an antibody, or antigen-binding portion thereof, that cross-competes for binding with CTLA-4 with at least one antibody having the amino acid of an antibody selected from the group consisting of 4.1.1, 4.8.1, 4.10.2, 4.13.1, 4.14.3, 6.1.1, 11.2.1, 11.6.1, 11.7.1., 12.3.1.1, 12.9.1.1, and 10D1.

9. The method of claim 1, wherein said antibody is a human antibody having the amino acid sequence of an antibody selected from the group consisting of 4.1.1, 4.13.1, 11.2.1, and 10D1.

10. The method of claim 9, wherein said antibody, or antigen-binding portion thereof, comprises a heavy chain and a light chain wherein the amino acid sequences of the heavy chain variable domain of said heavy chain and the light chain variable domain of said light chain are selected from the group consisting of:
(a) the amino acid sequence of SEQ ID NO:3 and the amino acid sequence of SEQ ID NO:9;
(b) the amino acid sequence of SEQ ID NO:15 and the amino acid sequence of SEQ ID NO:21;
(c) the amino acid sequence of SEQ ID NO:27 and the amino acid sequence of SEQ ID NO:33;
(d) the amino acid sequence encoded by the nucleic acid sequence of SEQ ID NO:1 and the amino acid sequence encoded by the nucleic acid sequence of SEQ ID NO:7;
(e) the amino acid sequence encoded by the nucleic acid sequence of SEQ ID NO:13 and the amino acid sequence encoded by the nucleic acid sequence of SEQ ID NO:19;
(f) the amino acid sequence encoded by the nucleic acid sequence of SEQ ID NO:25 and the amino acid sequence encoded by the nucleic acid sequence of SEQ ID NO:31; and
(g) the amino acid sequence of a variable domain of antibody 10D1.

11. The method of claim 9, wherein said antibody, or antigen-binding portion thereof, is an antibody selected from the group consisting of:
(a) an antibody comprising the amino acid sequences set forth in SEQ ID NO:4, SEQ ID NO:5, SEQ ID NO:6, SEQ ID NO:10, SEQ ID NO:11 and SEQ ID NO:12;
(b) an antibody comprising the amino acid sequences set forth in SEQ ID NO:16, SEQ ID NO:17, SEQ ID NO:18, SEQ ID NO:22, SEQ ID NO:23 and SEQ ID NO:24; and
(c) an antibody comprising the amino acid sequences set forth in SEQ ID NO:28, SEQ ID NO:29, SEQ ID NO:30, SEQ ID NO:34, SEQ ID NO:35 and SEQ ID NO:36.

12. The method of claim 9, wherein said antibody, or antigen-binding portion thereof, comprises a heavy chain variable region having the amino acid sequence set forth in SEQ ID NO:27 and a light chain variable region having the amino acid sequence set forth in SEQ ID NO:33.

13. The method of claim 9, wherein said antibody is selected from the group consisting of:
(a) an antibody comprising the amino acid sequences set forth in SEQ ID NO:2 and SEQ ID NO:8;
(b) an antibody comprising the amino acid sequences set forth in SEQ ID NO:14 and SEQ ID NO:20; and
(c) an antibody comprising the amino acid sequences set forth in SEQ ID NO:26 and SEQ ID NO:32.

14. The method of claim 1, wherein said antibody is administered 1-7 days prior to administration of said CpG ODN PF3512676.

15. The method of claim 1, wherein said CpG ODN PF3512676 is administered from about one to one-hundred days after said antibody.

16. The method of claim 1, wherein said CpG ODN PF3512676 is administered subcutaneously.

17. The method of claim 1, wherein said CpG ODN PF3512676 is administered in an amount of 1 mg-50 mg per day.

18. A pharmaceutical composition for treatment of cancer, said composition comprising a therapeutically effective amount of an anti-CTLA-4 antibody, or antigen-binding portion thereof, and a therapeutically effective amount of CpG ODN PF3512676, and a pharmaceutically acceptable carrier.
